SEQUENCE LISTING (28 sequences)

SEQ ID NO: 1 - DNA, 19 bp, Artificial Sequence
  chemically synthesized
atgcatgcatgcatgcatg

SEQ ID NO: 2 - protein, 26 aa, Artificial Sequence
  chemically synthesized
CFITKALGISYGRKKRRQRRRPPQGS

SEQ ID NO: 3 - protein, 12 aa, Artificial Sequence
  chemically synthesized
CMHIESLDSYTC

SEQ ID NO: 4 - protein, 12 aa, Artificial Sequence
  chemically synthesized
CMYIEALDKYAC

SEQ ID NO: 5 - DNA, 22 bp, Artificial Sequence
  chemically synthesized
ucccgcgacgaugccccucatt

SEQ ID NO: 6 - DNA, 22 bp, Artificial Sequence
  chemically synthesized
ugaggggcaucgucgcgggatt

SEQ ID NO: 7 - DNA, 21 bp, Artificial Sequence
  chemically synthesized
ucguccaggauggccgcggtt

SEQ ID NO: 8 - DNA, 21 bp, Artificial Sequence
  chemically synthesized
ccgcggccauccuggacgatt

SEQ ID NO: 9 - DNA, 21 bp, Artificial Sequence
  chemically synthesized
gugcaagugccaaccagactt

SEQ ID NO: 10 - DNA, 21 bp, Artificial Sequence
  chemically synthesized
gugugguuugcacuugcactt

SEQ ID NO: 11 - DNA, 21 bp, Artificial Sequence
  chemically synthesized
gugcaagugcaaaccagactt

SEQ ID NO: 12 - DNA, 21 bp, Artificial Sequence
  chemically synthesized
gucugguuugcacuugcactt

SEQ ID NO: 13 - RNA, 21 nt, Artificial Sequence
  chemically synthesized
gacguaaacggccacaaguuc

SEQ ID NO: 14 - RNA, 21 nt, Artificial Sequence
  chemically synthesized
acuuguggccguuuacgucgc

SEQ ID NO: 15 - DNA, 21 bp, Artificial Sequence
  chemically synthesized
cguacgcggaauacuucgatt

SEQ ID NO: 16 - DNA, 21 bp, Artificial Sequence
  chemically synthesized
ucgaaguauuccgcguacgtt

SEQ ID NO: 17 - RNA, 21 nt, Artificial Sequence
  chemically synthesized
gacguaaacggccacaaguuc

SEQ ID NO: 18 - RNA, 21 nt, Artificial Sequence
  chemically synthesized
acuuguggccguuuacgucgc

SEQ ID NO: 19 - DNA, 21 bp, Artificial Sequence
  chemically synthesized
gacgtaaacggccacaagttc

SEQ ID NO: 20 - DNA, 21 bp, Artificial Sequence
  chemically synthesized
gacgtaaacggccacaagttc

SEQ ID NO: 21 - DNA, 21 bp, Artificial Sequence
  chemically synthesized
gacgtaaacggccacaagttc

SEQ ID NO: 22 - DNA, 21 bp, Artificial Sequence
  chemically synthesized
cuuacgcugaguacuucgatt

SEQ ID NO: 23 - DNA, 21 bp, Artificial Sequence
  chemically synthesized
ucgaaguacucagcguaagtt

SEQ ID NO: 24 - RNA, 56 nt, Artificial Sequence
  chemically synthesized
gggaggacgaugcggaucagccauguuuacgucacuccuugucaauccucaucggc

SEQ ID NO: 25 - RNA, 23 nt, Artificial Sequence
  chemically synthesized
aacuuacgcugaguacuucgauu

SEQ ID NO: 26 - RNA, 79 nt, Artificial Sequence
  chemically synthesized
gggaggacgaugcggaucagccauguuuacgucacuccuugucaauccucaucggcaacuuacgcugaguacuucgauu

SEQ ID NO: 27 - RNA, 23 nt, Artificial Sequence
  chemically synthesized
aaucgaaguacucagcguaaguu

SEQ ID NO: 28 - RNA, 102 nt, Artificial Sequence
  chemically synthesized
gggaggacgaugcggaucagccauguuuacgucacuccuugucaauccucaucggcaacuuacgcugaguacuucgauuaaucgaaguacucagcguaaguu

(19) United States
(12) Patent Application Publication
(10) Pub. No.: US 2005/0256071 A1
(43) Pub. Date: Nov. 17, 2005

(54) Inhibitor nucleic acids

(75) Inventor: Mark E. Davis, Pasadena, CA (US)

Correspondence Address: FISH & NEAVE IP GROUP;ROPES & GRAY LLP, ONE INTERNATIONAL PLACE, BOSTON, MA, 02110-2624, US

(73) Assignee: California Institute of Technology, Pasadena, CA (US)

(21) Appl. No.: 11/044,677

(22) Filed: Jan. 27, 2005

Related U.S. Application Data
(63) Continuation-in-part of application No. 10/892,527, filed on Jul. 15, 2004.
(60) Provisional application No. 60/487,570, filed on Jul. 15, 2003.
(60) Provisional application No. 60/528,143, filed on Dec. 8, 2003.

(51) Int. Cl. A61K 48/00; C07H 21/02

(52) U.S. Cl. 514/44; 536/23.1

(57) ABSTRACT

The present invention provides methods and compositions for attenuating expression of a target gene in vivo. In general, the method includes administering RNAi constructs (such as small-interfering RNAs (i.e., siRNAs) that are targeted to particular mRNA sequences, or nucleic acid material that can produce siRNAs in a cell), in an amount sufficient to attenuate expression of a target gene by an RNA interference mechanism. In particular, the RNAi constructs may include one or more modifications to improve serum stability, cellular uptake and/or to avoid non-specific effect. In certain embodiments, the RNAi constructs contain an aptamer portion. The aptamer may bind to human serum albumin to improve serum half life. The aptamer may also bind to a cell surface protein that improves uptake of the construct.

### CROSS-REFERENCE TO RELATED APPLICATIONS

This application is a Continuation-in-Part of U.S. application Ser. No. 10/892,527, filed July 15, 2004, which claims the benefit of the filing date of U.S. Provisional Application No. 60/487,570, filed Jul. 15, 2003, and of U.S. Provisional Application No. 60/528,143, filed Dec. 8, 2003, the specifications of which are incorporated by reference herein in their entirety.

### BACKGROUND OF THE INVENTION

The structure and biological behavior of a cell is determined in large part by the pattern of gene expression within that cell at a given time. Perturbations of gene expression have long been acknowledged to account for a vast number of diseases including numerous forms of cancer, vascular diseases, neuronal and endocrine diseases. Abnormal expression patterns, caused, for example, by amplification, deletion, gene rearrangements, and loss or gain of function mutations, are now known to lead to aberrant behavior of a disease cell. Aberrant gene expression has also been noted as a defense mechanism of certain organisms to ward off the threat of pathogens.

One of the major challenges of medicine has been to regulate the expression of targeted genes that are implicated in a wide diversity of physiological responses. While over-expression of an exogenously introduced transgene in a eukaryotic cell is relatively straightforward, targeted inhibition of specific genes has been more difficult to achieve. Traditional approaches for suppressing gene expression, including site-directed gene disruption, antisense RNA or co-suppression, require complex genetic manipulations or heavy dosages of suppressors that often exceed the toxicity tolerance level of the host cell.

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules. Gil et al. Apoptosis 2000, 5:107-114. The field was significantly advanced upon the demonstration that synthetic duplexes of 21 nucleotide RNAs could mediate gene specific RNAi in mammalian cells, without invoking generic antiviral defense mechanisms. Elbashir et al. Nature 2001, 411:494-498; Caplen et al. Proc Natl Acad Sci 2001, 98:9742-9747. As a result, small-interfering RNAs (siRNAs) have become powerful tools to dissect gene function. The chemical synthesis of small RNAs is one avenue that has produced promising results.

Methods for delivering RNAi nucleic acids in vivo have been difficult to develop. It would be desirable to have improved methods and compositions for the administration of RNAi molecules in a clinical setting. More specifically, it would be desirable to have improved siRNA molecules that would not induce undesirable, non-specific side effects. It would also be desirable to have siRNA molecules having improved stability in serum and exhibiting increased uptake by animal cells.

### SUMMARY OF THE INVENTION

The invention provides, in part, novel RNAi constructs. In certain aspects, the invention provides nucleic acid RNAi constructs, optionally comprising one or more modifications. In certain aspects, the novel constructs disclosed herein have one or more improved qualities relative to traditional RNA:RNA RNAi constructs, including, for example, improved serum stability, or improved cellular uptake. In certain aspects, an RNAi construct is attached to an aptamer that provides desirable properties and/or functionalities, including, for example, the ability to bind to serum proteins or proteins located on target cells. In yet further aspects, a construct disclosed herein may include a component, such as a mismatch or a denaturant, that reduces the melting point for the duplex.

The invention provides, in part, RNAi constructs comprising one or more chemical modifications that enhance serum stability and/or cellular uptake of the constructs. In certain embodiments, the RNAi constructs disclosed herein have improved cellular uptake in vivo, relative to unmodified RNAi constructs. In certain embodiments, the RNAi constructs disclosed herein have a longer serum half-life relative to unmodified RNAi constructs. In certain aspects, the chemical modifications may be selected so as to increase the noncovalent association of an RNAi construct with one or more proteins. In general, a modification that decreases the overall negative charge and/or increases the hydrophobicity of an RNAi construct will tend to increase noncovalent association with proteins. In a preferred embodiment, the modifications are incorporated into the sense strand of a double-stranded RNAi construct. A modification may be in the form of a chemical moiety, such as a hydrophobic moiety, which is conjugated to a nucleic acid of the RNAi construct. A modification may also be in the form of an alteration to the nucleic acid itself, such as an alteration to the sugar-phosphate backbone or to the base portion.

In certain embodiments, the invention provides a double-stranded nucleic acid having a designated sequence for inhibiting target gene expression by an RNAi mechanism, comprising: a sense polynucleotide strand having one or more modifications; and an RNA antisense polynucleotide strand having a designated sequence that hybridizes to at least a portion of a transcript of the target gene and is sufficient for silencing the target gene. The one or more modifications of the sense and/or antisense strand may increase non-covalent association of the double-stranded nucleic acid with one or more species of protein as compared to an unmodified double-stranded nucleic acid having the same designated sequence. Modifications may be modifications of the sugar-phosphate backbone. Modifications may also be modification of the nucleoside portion. Optionally, the sense strand is a DNA or RNA strand comprising 10%, 20%, 30%, 40%, 50%, 60%, 70%, 80%, 90% or 100% modified nucleotides. Optionally, the sense polynucleotide is a DNA strand comprising one or more modified deoxyribonucleotides. Optionally, the sense polynucleotide is an RNA strand comprising a plurality of modified ribonucleotides. Optionally, the sense polynucleotide is an XNA strand, such as a peptide nucleic acid (PNA) strand or locked nucleic acid (LNA) strand. Optionally the RNA antisense strand comprises one or more modifications. For example, the RNA antisense strand may comprise no more than 10%, 20%, 30%, 40%, 50% or 75% modified nucleotides. The one or more modifications may be selected so as increase the hydrophobicity and/or stability (to nucleases, for example) of the double-stranded nucleic acid, in physiological conditions, relative to an unmodified double-stranded nucleic acid having the same designated sequence.

In certain embodiments, the invention provides for RNAi constructs and formulations that bind to one or more target proteins. For example, RNAi constructs may be formulated with or conjugated to one or more proteins (e.g. antibodies) that bind to a target protein. As another example, an RNAi construct may comprise one or more aptamers or may be noncovalently formulated with one or more aptamers. An aptamer is a nucleic acid that interacts with a target of interest to form an aptamer:target complex. The aptamer may be incorporated into or be attached to either the sense or antisense strand and may occur at either the 3′ or 5′ end of either strand, although it is expected that aptamers positioned at the 5′ end of the sense strand will tend to have fewer detrimental effects on the RNAi activity of the construct. Incorporation or attachment of the aptamer to the sense or antisense strand allows each component to retain its activity; that is, the aptamer component retains the ability to interact with a specific target, and the sense and/or antisense strands retain their ability to inhibit target gene expression by an RNAi mechanism. In some embodiments, the aptamer may be selected from a plurality of aptamers (e.g. from a nucleic acid library) which may have been screened and/or optimized to impute a beneficial property onto the system, such as binding to a particular target. The aptamers of the present invention may be chemically synthesized and developed in vitro through the SELEX screening process. The aptamer may be chosen to preferentially interact with and/or bind to a target. Suitable categories of such targets include molecules, such as small organic molecules, nucleotides, polynucleotides, peptides, polypeptides, and proteins. Other targets include larger structures such as organelles, viruses, and cells. Examples of suitable proteins include extracellular proteins, membrane proteins, cell surface proteins, or serum proteins (e.g. an albumin such as human serum albumin). Such target molecules may be internalized by a cell. Interaction of the aptamer with the target molecule (e.g. peptide, protein, etc.) may improve bioavailability and/or cellular uptake of the aptamer and/or polynucleotide. The aptamer and/or polynucleotide may be internalized by a cell, and binding of the aptamer to a target molecule, such as a peptide, polypeptide, or protein, may facilitate internalization of the polynucleotide into the cell. Modifications that may be made to the polynucleotides of the instant invention may also be made to one or more aptamers. It will be understood that a RNAi construct may comprise an aptamer in situations where the sense or antisense portions of the RNAi construct also participate in target binding activity. In other words, the present disclosure further provides RNAi constructs where the “aptamer” or target-binding portion of the construct overlaps the sense or antisense portion of the construct.

In certain embodiments, the RNAi construct comprising the one or more modifications has a log P value at least 0.5 log P units less than the log P value of an otherwise identical unmodified RNAi construct, and preferably at least 1, 2, 3 or even 4 log P unit less than the log P value of an otherwise identical unmodified RNAi construct. The one or more modifications may be selected so as increase the positive charge (or decrease the negative charge) of the double-stranded nucleic acid, in physiological conditions, relative to an unmodified double-stranded nucleic acid having the same designated sequence. In certain embodiments, the RNAi construct comprising the one or more modifications has an isoelectric pH (pI) that is at least 0.25 units higher than the otherwise identical unmodified RNAi construct, and preferably at least 0.5, 1 or even 2 units higher than the otherwise identical unmodified RNAi construct. Optionally, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety and a 2′-deoxy-2′-fluoride moiety. Optionally, the sense polynucleotide is covalently bonded to a hydrophobic moiety, which may be attached, for example, to the 3′- or 5′-terminus or the sugar-phosphate backbone or the nucleoside portion. In certain embodiments, the RNAi construct is a hairpin nucleic acid that is processed to an siRNA inside a cell. The length of each strand of the double-stranded nucleic acid may be selected so as to avoid provoking a clinically unacceptable inflammatory response. Optionally, each strand of the double-stranded nucleic acid may be 19-100 base pairs long, and preferably 19-50 or 19-30 base pairs long (not including aptamer modifications). It is generally expected that nucleotides of 29 bases or fewer will not provoke an inflammatory response, while longer nucleotides may need to be evaluated for inflammatory effects on a case-by-case basis.

In certain embodiments, a double-stranded RNAi construct disclosed herein is internalized by cultured cells in the presence of 10% serum to a steady state level that is at least twice that of the unmodified double-stranded nucleic acid having the same designated sequence, and preferably the level of internalized modified RNAi construct is at least three, five or about ten times higher than for the unmodified form.

In certain embodiments, a double-stranded RNAi construct disclosed herein has a serum half-life in a human or mouse of at least twice that of the unmodified double-stranded nucleic acid having the same designated sequence and optionally the serum half-life of the modified RNAi construct is at least three or five times higher than for the unmodified form.

In certain embodiments, the RNAi construct comprising one or more modifications has a K_{D} for a selected protein that is at least 0.2 log units less than the K_{D} of the otherwise identical unmodified RNAi construct, and preferably at least 0.5 or 1.0 units less than the K_{D} of the otherwise identical unmodified construct for the same selected protein. In other words, the RNAi construct may be designed so as to have an increased affinity for a selected protein.

In certain embodiments, the RNAi construct comprising one or more modifications has an ED50 for producing the clinical response at least 2 times less than the ED50 of the otherwise identical unmodified RNAi construct, and even more preferably at least 5 or 10 times less. In other words, the RNAi construct comprising one or more modification may have a therapeutic effect at lower dosage levels.

In certain embodiments, the invention provides an RNAi construct comprising a double-stranded nucleic acid, wherein the sense strand or the antisense strand includes one or more modifications. In a preferred embodiment, the sense strand comprises one or more modifications, optionally greater than 50%, greater than 80% or even 100% modified nucleotides, while the antisense strand comprises only unmodified nucleotides. The modifications of the sense strand may be selected so as to enhance the serum stability and/or cellular uptake of the RNAi construct. For example, the sense strand may comprise phosphorothioate modifications, optionally at greater than 50%, greater than 80% or even at 100% of the available positions for such modifications. As evidenced by the examples herein, an RNA:RNA construct in which the sense strand comprises 100% phosphorothioate moieties is highly effective for delivery in vivo. In certain embodiments, the double-stranded nucleic acid comprises mismatched base pairs. In certain embodiments, the RNAi nucleic acid has a Tm lower than the Tm of a double-stranded nucleic acid comprising the same antisense strand complemented by a perfectly matched sense strand. The Tm comparison is based on Tms of the nucleic acids under the same ionic strength and preferably, physiological ionic strength. The Tm may be lower by 1° C., 2° C., 3° C., 4° C., 5° C., 10° C., 15° C., or 20° C.

In certain aspects, the invention provides pharmaceutical preparations for delivery to a subject comprising RNAi constructs with one or more modified nucleic acids. In some embodiments, a pharmaceutical preparation comprises a double-stranded nucleic acid having a designated sequence for inhibiting target gene expression by an RNAi mechanism, comprising: a sense polynucleotide strand having one or more modifications; and an RNA antisense polynucleotide strand optionally comprising one or more modifications or modified nucleotides and having a designated sequence that hybridizes to at least a portion of a transcript of the target gene and is sufficient for silencing the target gene. The one or more modifications of the sense and/or antisense strand increase non-covalent association of the double-stranded nucleic acid with one or more species of protein as compared to an unmodified double-stranded nucleic acid having the same designated sequence. Modifications may be modifications of the sugar-phosphate backbone, such as phosphorothioate modifications. Modifications may also be modifications of the nucleoside portion. Optionally, the sense strand is a DNA or RNA strand comprising 10%, 20%, 30%, 40%, 50%, 60%, 70%, 80%, 90% or 100% modified nucleotides. Optionally, the sense polynucleotide is a DNA strand comprising one or more modified deoxyribonucleotides. Optionally, the sense polynucleotide is an RNA strand comprising a plurality of modified ribonucleotides. Optionally, the sense polynucleotide is an XNA strand, such as a peptide nucleic acid (PNA) strand or locked nucleic acid (LNA) strand. Optionally the RNA antisense strand comprises one or more modifications. For example, the RNA antisense strand may comprise no more than 10%, 20%, 30%, 40%, 50% or 75% modified nucleotides. The one or more modifications may be selected so as increase the hydrophobicity and/or stability (to nucleases, for example) of the double-stranded nucleic acid, in physiological conditions, relative to an unmodified double-stranded nucleic acid having the same designated sequence.

In instances where an RNAi construct includes an aptamer, modifications of the polynucleotide strands of the RNAi construct may be positioned within the aptamer portion. For example, modifications that increase the hydrophobicity or decrease the charge of an RNAi construct may be positioned within the aptamer portion, so long as such modifications are consistent with target binding activity.

In certain embodiments, the RNAi construct comprising the one or more modifications has a log P value at least 0.5 log P units less than the log P value of an otherwise identical unmodified RNAi construct, and preferably at least 1, 2, 3 or even 4 log P unit less than the log P value of an otherwise identical unmodified RNAi construct. The one or more modifications may be selected so as increase the positive charge (or decrease the negative charge) of the double-stranded nucleic acid, in physiological conditions, relative to an unmodified double-stranded nucleic acid having the same designated sequence. In certain embodiments, the RNAi construct comprising the one or more modifications has an isoelectric pH (pI) that is at least 0.25 units higher than the otherwise identical unmodified RNAi construct, and preferably at least 0.5, 1 or even 2 units higher than the otherwise identical unmodified RNAi construct. Optionally, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety and a 2′-deoxy-2′-fluoride moiety. In certain embodiments, the RNAi construct is a hairpin nucleic acid that is processed to an siRNA inside a cell. Optionally, each strand of the double-stranded nucleic acid may be 19-100 base pairs long, and preferably 19-50 or 19-30 base pairs long (not including aptamer modifications).

In certain embodiments, the invention provides pharmaceutical preparations comprising the RNAi constructs disclosed herein. A pharmaceutical preparation may further comprise a polypeptide, such as a polypeptide selected from amongst serum polypeptides, cell targeting polypeptides and internalizing polypeptides. Examples of cell targeting polypeptides include a polypeptide comprising a plurality of galactose moieties for targeting to hepatocytes (e.g., asialoglycoproteins, such as asialofetuin), a transferrin polypeptide for targeting to neoplastic cells and an antibody that binds selectively to a cell of interest. A polypeptide may be associated with the RNAi constructs, covalently or non-covalently.

In preferred embodiments, a pharmaceutical preparation of the invention comprises an RNAi construct comprising a double-stranded nucleic acid, wherein the sense strand includes one or more modifications and wherein the antisense strand is an RNA strand. The modifications of the sense strand may be selected so as to enhance the serum stability and/or cellular uptake of the RNAi constructs. In certain embodiments, the double-stranded nucleic acid comprises mismatched base pairs. In certain embodiments, the RNAi nucleic acid under physiological ionic strength has a Tm lower than the Tm of a double-stranded nucleic acid comprising the same RNA antisense strand complemented by a perfectly matched sense strand under physiological ionic strength.

In certain embodiments, a pharmaceutical preparation for delivery to a subject may comprise an RNAi construct of the invention and a pharmaceutically acceptable carrier. Optionally, the pharmaceutically acceptable carrier is selected from pharmaceutically acceptable salts, ester, and salts of such esters. A pharmaceutical preparation may be packaged with instructions for use with a human or other animal patient.

In certain embodiments, the disclosure provides methods for decreasing the expression of a target gene in a cell, the method comprising contacting the cell with a composition comprising a double-stranded nucleic acid, the double-stranded nucleic acid comprising: a sense polynucleotide strand comprising one or more modifications; and an RNA antisense polynucleotide strand optionally comprising one or more modifications or modified nucleotides and having a designated sequence that hybridizes to at least a portion of a transcript of the target gene and is sufficient for silencing the target gene, wherein the one or more modifications increase, relative to an unmodified double-stranded nucleic acid having the designated sequence, serum stability and/or cellular uptake of the RNAi construct.

Optionally, the cell is contacted with the double-stranded nucleic acid in the presence of at least 0.1 milligram/milliliter of protein and preferably at least 0.5, 1, 2 or 3 milligrams per milliliter. Optionally, the cell is contacted with the double-stranded nucleic acid in the presence of serum, such as at least 1%, 5%, 10%, or 15% serum. Optionally, the cell is contacted with the double-stranded nucleic acid in the presence of a protein concentration that mimics a physiological concentration.

In certain embodiments, the disclosure provides methods for decreasing the expression of a target gene in one or more cells of a subject, the method comprising administering to the subject a composition comprising a double-stranded nucleic acid, the double-stranded nucleic acid comprising: a sense polynucleotide strand comprising one or more modifications; and an RNA antisense polynucleotide strand optionally comprising one or more modifications or modified nucleotides and having a designated sequence that hybridizes to at least a portion of a transcript of the target gene and is sufficient for silencing the target gene, wherein the one or more modifications increase, relative to an unmodified double-stranded nucleic acid having the designated sequence, serum stability and/or cellular uptake of the RNAi construct. In certain embodiments, the double-stranded nucleic acid comprises mismatched base pairs. In certain embodiments, the double-stranded nucleic acid under physiological ionic strength has a Tm lower than the Tm of a double-stranded nucleic acid comprising the same RNA antisense strand complemented by a perfectly matched sense strand.

In some embodiments, a method disclosed herein employs a double-stranded nucleic acid having a designated sequence for inhibiting target gene expression by an RNAi mechanism, comprising: a sense polynucleotide strand having one or more modifications; and an RNA antisense polynucleotide strand optionally comprising one or more modifications or modified nucleotides and having a designated sequence that hybridizes to at least a portion of a transcript of the target gene and is sufficient for silencing the target gene. The one or more modifications of the sense and/or antisense strand may be selected so as to increase non-covalent association of the double-stranded nucleic acid with one or more species of protein as compared to an unmodified double-stranded nucleic acid having the same designated sequence. Modifications may be selected, empirically or otherwise, so as to enhance cellular uptake and/or serum stability. Modifications may be modifications of the sugar-phosphate backbone. Modifications may also be modification of the nucleoside portion. Optionally, the sense strand is a DNA or RNA strand comprising 10%, 20%, 30%, 40%, 50%, 60%, 70%, 80%, 90% or 100% modified nucleotides. Optionally, the sense polynucleotide is a DNA strand comprising one or more modified deoxyribonucleotides. Optionally, the sense polynucleotide is an RNA strand comprising a plurality of modified ribonucleotides. Optionally, the sense polynucleotide is an XNA strand, such as a peptide nucleic acid (PNA) strand or locked nucleic acid (LNA) strand. Optionally the RNA antisense strand comprises one or more modifications. For example, the RNA antisense strand may comprise no more than 10%, 20%, 30%, 40%, 50% or 75% modified nucleotides. The one or more modifications may be selected so as increase the hydrophobicity and/or stability (to nucleases, for example) of the double-stranded nucleic acid, in physiological conditions, relative to an unmodified double-stranded nucleic acid having the same designated sequence. In certain embodiments, the RNAi construct comprising the one or more modifications has a log P value at least 0.5 log P units less than the log P value of an otherwise identical unmodified RNAi construct, and preferably at least 1, 2, 3 or even 4 log P unit less than the log P value of an otherwise identical unmodified RNAi construct. The one or more modifications may be selected so as increase the positive charge (or increase the negative charge) of the double-stranded nucleic acid, in physiological conditions, relative to an unmodified double-stranded nucleic acid having the same designated sequence. In certain embodiments, the RNAi construct comprising the one or more modifications has an isoelectric pH (pI) that is at least 0.25 units higher than the otherwise identical unmodified RNAi construct, and preferably at least 0.5, 1 or even 2 units higher than the otherwise identical unmodified RNAi construct. Optionally, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety and a 2′-deoxy-2′-fluoride moiety. In certain embodiments, the double stranded nucleic acid is a hairpin nucleic acid that is processed to an siRNA inside a cell. Optionally, each strand of the double-stranded nucleic acid may be 19-100 base pairs long, and preferably 19-50 or 19-30 base pairs long (not including aptamer modifications). Optionally, the double stranded nucleic acid comprises an aptamer.

In certain embodiments, a composition employed in a disclosed method further comprises a polypeptide, such as a polypeptide selected from amongst serum polypeptides, cell targeting polypeptides and internalizing polypeptides. Examples of cell targeting polypeptides include a polypeptide comprising a plurality of galactose moieties for targeting to hepatocytes, a transferrin polypeptide for targeting to neoplastic cells and an antibody that binds selectively to a cell of interest.

In certain embodiments, the disclosure provides coatings for use on surface of a medical device. A coating may comprise a polymer matrix having RNAi constructs dispersed therein, which RNAi constructs are eluted from the matrix when implanted at site in a patient's body and alter the growth, survival or differentiation of cells in the vicinity of the implanted device. In certain embodiments, at least one of the RNAi constructs is a double-stranded nucleic acid comprising: a sense polynucleotide strand comprising one or more modifications; and an RNA antisense polynucleotide strand optionally comprising one or more modifications or modified nucleotides and having a designated sequence that hybridizes to at least a portion of a transcript of the target gene and is sufficient for silencing the target gene, wherein the one or more modifications increase, relative to an unmodified double-stranded nucleic acid having the designated sequence, serum stability and/or cellular uptake of the RNAi construct. A coating may further comprise a polypeptide. A coating may be situated on the surface of a variety of medical devices, including, for example, a screw, plate, washers, suture, prosthesis anchor, tack, staple, electrical lead, valve, membrane, catheter, implantable vascular access port, blood storage bag, blood tubing, central venous catheter, arterial catheter, vascular graft, intraaortic balloon pump, heart valve, cardiovascular suture, artificial heart, pacemaker, ventricular assist pump, extracorporeal device, blood filter, hemodialysis unit, hemoperfasion unit, plasmapheresis unit, and filter adapted for deployment in a blood vessel. Preferably the coating is on a surface of a stent.

In some embodiments, a coating disclosed herein includes a double-stranded nucleic acid having a designated sequence for inhibiting target gene expression by an RNAi mechanism, comprising: a sense polynucleotide strand having one or more modifications; and an RNA antisense polynucleotide strand optionally comprising one or more modifications or modified nucleotides and having a designated sequence that hybridizes to at least a portion of a transcript of the target gene and is sufficient for silencing the target gene. The one or more modifications of the sense and/or antisense strand increase non-covalent association of the double-stranded nucleic acid with one or more species of protein as compared to an unmodified double-stranded nucleic acid having the same designated sequence. Modifications may be selected so as to increase serum stability and/or cellular uptake. Modifications may be modifications of the sugar-phosphate backbone. Modifications may also be modification of the nucleoside portion. Optionally, the sense strand is a DNA or RNA strand comprising 10%, 20%, 30%, 40%, 50%, 60%, 70%, 80%, 90% or 100% modified nucleotides. Optionally, the sense polynucleotide is a DNA strand comprising one or more modified deoxyribonucleotides. Optionally, the sense polynucleotide is an RNA strand comprising a plurality of modified ribonucleotides. Optionally, the sense polynucleotide is an XNA strand, such as a peptide nucleic acid (PNA) strand or locked nucleic acid (LNA) strand. Optionally the RNA antisense strand comprises one or more modifications. For example, the RNA antisense strand may comprise no more than 10%, 20%, 30%, 40%, 50% or 75% modified nucleotides. The one or more modifications may be selected so as increase the hydrophobicity and/or stability (to nucleases, for example) of the double-stranded nucleic acid, in physiological conditions, relative to an unmodified double-stranded nucleic acid having the same designated sequence. In certain embodiments, the RNAi construct comprising the one or more modifications has a log P value at least 0.5 log P units less than the log P value of an otherwise identical unmodified RNAi construct, and preferably at least 1, 2, 3 or even 4 log P unit less than the log P value of an otherwise identical unmodified RNAi construct. The one or more modifications may be selected so as increase the positive charge (or increase the negative charge) of the double-stranded nucleic acid, in physiological conditions, relative to an unmodified double-stranded nucleic acid having the same designated sequence. In certain embodiments, the RNAi construct comprising the one or more modifications has an isoelectric pH (pI) that is at least 0.25 units higher than the otherwise identical unmodified RNAi construct, and preferably at least 0.5, 1 or even 2 units higher than the otherwise identical unmodified RNAi construct. Optionally, the sense polynucleotide comprises a modification to the phosphate-sugar backbone selected from the group consisting of: a phosphorothioate moiety, a phosphoramidate moiety, a phosphodithioate moiety, a PNA moiety, an LNA moiety, a 2′-O-methyl moiety and a 2′-deoxy-2′-fluoride moiety. In certain embodiments, the RNAi construct is a hairpin nucleic acid that is processed to an siRNA inside a cell. Optionally, each strand of the double-stranded nucleic acid may be 19-100 base pairs long, and preferably 19-50 or 19-30 base pairs long (not including aptamer modifications).

In certain embodiments, a coating disclosed herein may comprise a polypeptide that associates with the RNAi construct, such as a polypeptide selected from amongst serum polypeptides, cell targeting polypeptides and internalizing polypeptides. Examples of cell targeting polypeptides include a polypeptide comprising a plurality of galactose moieties for targeting to hepatocytes, a transferrin polypeptide for targeting to neoplastic cells and an antibody that binds selectively to a cell of interest.

In certain aspects, the disclosure provides methods of optimizing RNAi constructs for pharmaceutical uses, involving evaluating cellular uptake and/or pharmacokinetic properties (e.g., serum half-life) of RNAi constructs comprising one or more modified nucleic acids. In certain embodiments, a method of optimizing RNAi constructs for pharmaceutical uses comprises: identifying an RNAi construct having a designated sequence which inhibits the expression of a target gene in vivo and reduces the effects of a disorder; designing one or more modified RNAi constructs having the designated sequence and comprising one or more modified nucleic acids; testing the one or more modified RNAi constructs for uptake into cells and/or serum half-life; conducting therapeutic profiling of the modified and/or unmodified RNAi constructs of for efficacy and toxicity in animals; selecting one or more modified RNAi constructs having desirable uptake properties and desirable therapeutic properties. In certain embodiments, the method comprises replacing the sense strand of an identified RNAi construct with a sense strand that may comprise one or more modifications or modified nucleotides. In certain embodiments, the method of optimizing RNAi constructs for pharmaceutical uses comprises generating a plurality of test RNAi constructs comprising a double-stranded nucleic acid and testing for gene silencing effects by these test constructs. The sense and/or antisense strand of the nucleic acid may comprise one or more modifications or modified nucleotides. The double-stranded nucleic acid may comprise one or more mismatched base pairs. The method may further comprise determining serum stability and/or cellular uptake of the test RNAi constructs and conducting therapeutic profiling of the test RNAi constructs.

The methods of optimizing RNAi constructs for pharmaceutical uses may further comprise formulating a pharmaceutical preparation including one or more of the selected RNAi constructs. Optionally, the methods may further comprise any of the following: establishing a distribution system for distributing the pharmaceutical preparation for sale, partnering with another corporate entity to effect distribution, establishing a sales group for marketing the pharmaceutical preparation, and establishing a profitable reimbursement program with one or more private or government health care insurers.

### BRIEF DESCRIPTION OF DRAWINGS

FIG. 1 is a photograph of a gel showing amount of nucleic acids under conditions indicated as follows:

| | |
|---|---|
| Lane 1 | siFAS2, H2O |
| Lane 2 | siFAS2, serum (t = 0) |
| Lane 3 | siFAS2, serum (t = 4 h) |
| Lane 4 | CDP/siFAS2 5 +/−, serum (t = 4 h), no heparan sulfate |
| Lane 5 | CDP/siFAS2 5 +/−, serum (t = 4 h), heparan sulfate |
| Lane 6 | [hybrid], H2O |
| Lane 7 | [hybrid], serum (t = 0) |
| Lane 8 | [hybrid], serum (t = 4 h) |
| Lane 9 | CDP/[hybrid] 5 +/−, serum (t = 4 h), no heparan sulfate |
| Lane 10 | CDP/[hybrid] 5 +/−, serum (t = 4 h), heparan sulfate |

wherein [hybrid] = JH-1: EGFPb-anti = DNA(PS)-3′TAMRAs: RNAa

FIG. 2 is a photograph of a gel showing amount of nucleic acids under conditions indicated as follows:

| | |
|---|---|
| Lane 1 | 10 bp DNA ladder |
| Lane 2 | siFAS2, serum H2O |
| Lane 3 | siFAS2, serum (t = 0) |
| Lane 4 | siFAS2, serum (t = 4 h) |
| Lane 5 | CDP/siFAS2 5 +/−, serum (t = 4 h), no heparan sulfate |
| Lane 6 | CDP/siFAS2 5 +/−, serum (t = 4 h), heparan sulfate |
| Lane 7 | CDP/siFAS2 10 +/−, serum (t = 4 h), no heparan sulfate |
| Lane 8 | CDP/siFAS2 10 +/−, serum (t = 4 h), heparan sulfate |
| Lane 9 | CDP/siFAS2 20 +/−, serum (t = 4 h), no heparan sulfate |
| Lane 10 | CDP/siFAS2 20 +/−, serum (t = 4 h), heparan sulfate |

FIG. 3A-3D show confocal microscopy results demonstrating in vivo uptake of nucleic acid constructs.

FIG. 4 shows a schematic for the animal model experiment.

FIG. 5A-B show the results of delivery of a modified siRNA in a mouse.

FIG. 6 shows the predicted secondary structure for the xPSM-A10-3 aptamer.

FIG. 7A-B show the predicted two most thermodynamically favorable secondary structures for the xPSM-A10-3-SiGL3 aptamer-siRNA conjugate.

### DETAILED DESCRIPTION OF THE INVENTION

I. Overview

In certain aspects, the present invention relates to the finding that certain modifications improve serum stability and facilitate the cellular uptake of RNAi constructs. Another aspect of the present invention relates to optimizing RNAi constructs to avoid non-specific, “off-target” effects, e.g., effects induced by the sense RNA strand of an RNA:RNA siRNA molecule, or possibly effects related to RNA-activated protein kinase (“PKR”) and interferon response. Accordingly, in certain aspects, the invention provides modified double stranded RNAi constructs for use in decreasing the expression of target genes in cells, particularly in vivo. Traditional, naked antisense molecules can be effectively administered into animals and humans. However, typical RNAi constructs, such as short double-stranded RNAs, are not so easily administered. In addition, a discrepancy has been observed between the effectiveness of RNAi delivery to cells during in vitro experiments versus in vivo experiments. As demonstrated herein, chemical or biological modifications of an RNAi construct improve serum stability of the RNAi construct. The modifications further facilitate the uptake of the RNAi construct by a cell. In part, the present disclosure demonstrates that unmodified RNAi constructs tend to have poor serum stability and be taken up poorly. As shown in the appended examples, constructs of the invention demonstrate increased serum stability and improved in vivo uptake. While not wishing to be bound by any particular theory, an improved RNAi construct without a double-stranded RNA:RNA siRNA may avoid the non-specific effect induced by double-stranded RNA:RNA siRNAs, e.g., the off-target effect induced by the sense strand RNA of an RNA:RNA siRNA molecule. Thus, the present invention provides double-stranded nucleic acid RNAi constructs comprising nucleic acids having mismatched base pairs.

Accordingly, the invention provides, in part, RNAi constructs comprising a nucleic acid that has been modified so as to increase its serum stability and/or cellular uptake. The nucleic acid may be further improved to avoid non-specific effects.

II. Definitions

For convenience, certain terms employed in the specification, examples, and appended claims are collected here.

The term “aptamer” includes any nucleic acid sequence that is capable of specifically interacting with a target. An aptamer may be a naturally occurring nucleic acid sequence or a nucleic acid sequence that is not naturally occurring. Aptamers may be any type of nucleic acid (e.g. DNA, RNA or nucleic acid analogs) and may be single-stranded or double-stranded. In certain specific embodiments described herein, aptamers are a single-stranded RNA.

An “aptamer:target complex” or “aptamer:target molecule complex” is a complex comprising an aptamer and the target or target molecule with which it interacts. The aptamer and the target or target molecule need not be directly bound to each other.

A “patient” or “subject” to be treated by a disclosed method can mean either a human or non-human animal.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence. A method that decreases the expression of a gene may do so in a variety of ways (none of which are mutually exclusive), including, for example, by inhibiting transcription of the gene, decreasing the stability of the mRNA and decreasing translation of the mRNA. While not wishing to be bound to a particular mechanism, it is generally thought that siRNA techniques decrease gene expression by stimulating the degradation of targeted mRNA species.

By “silencing” a target gene herein is meant decreasing or attenuating the expression of the target gene.

As used herein, the term “nucleic acid” refers to polynucleotides such as deoxyribonucleic acid (DNA) and ribonucleic acid (RNA). The term should also be understood to include, as applicable to the embodiment being described, single-stranded (such as sense or antisense) and double-stranded polynucleotides. The “canonical” nucleotides are adenosine (A), guanosine (G), cytosine (C), thymidine (T), and uracil (U), and include a ribose-phosphate backbone, but the term nucleic acid is intended to include polynucleotides comprising only canonical nucleotides as well as polynucleotides including one or more modifications to the sugar phosphate backbone or the nucleoside. DNA and RNA are chemically different because of the absence or presence of a hydroxyl group at the 2′ position on the ribose. Modified nucleic acids that cannot be readily termed DNA or RNA (e.g. in which an entirely different moiety is positioned at the 2′ position) and nucleic acids that do not contain a ribose-based backbone may be referred to as XNAs. Examples of XNAs are peptide nucleic acids (PNAs) in which the backbone is a peptide backbone, and locked nucleic acids (LNAs) containing a methylene linkage between the 2′ and 4′ positions of the ribose. An “unmodified” nucleic acid is a nucleic acid that contains only canonical nucleotides and a DNA or RNA backbone. For clarification, it will be apparent to one of skill in the field that nucleic acids will often have both single-stranded and double-stranded portions and that such portions may form and dissociate in different conditions. As the term is used herein, a “double-stranded” nucleic acid is any nucleic acid that comprises a double-helical portion under physiological conditions.

A “nucleic acid library” is any collection of a plurality of nucleic acid species (nucleic acids having different sequences) The nucleic acids of a library are often but not always, situated in vectors, with one nucleic acid species (or “insert”)/per vector.

The term “pharmaceutically acceptable salts” refers to physiologically and pharmaceutically acceptable salts of the compounds of the invention, i.e., salts that retain the desired biological activity of the parent compound and do not impart undesired toxicological effects thereto.

The terms “polypeptide” and “protein” are used interchangeably herein.

The terms “pulmonary delivery” and “respiratory delivery” refer to systemic delivery of RNAi constructs to a patient by inhalation through the mouth and into the lungs.

As used herein, the term “RNAi construct” is a generic term used throughout the specification to include small interfering RNAs (siRNAs), hairpin RNAs, and other RNA species which can be cleaved in vivo to form siRNAs. Optionally, the siRNA include single strands or double strands, including DNA:RNA, RNA:RNA and XNA:RNA double-stranded nucleic acids.

The term “small interfering RNAs” or “siRNAs” refers to nucleic acids around 19-30 nucleotides in length, and more preferably 21-23 nucleotides in length. The siRNAs are double-stranded, and may include short overhangs at each end. While the antisense strand of a siRNA is preferably RNA, the sense strand may be RNA, DNA or XNA, as well as modifications and mixtures thereof. Preferably, the overhangs are 1-6 nucleotides in length at the 3′ end. It is known in the art that the siRNAs can be chemically synthesized, or derive from a longer double-stranded RNA or a hairpin RNA. The siRNAs have significant sequence similarity to a target RNA so that the siRNAs can pair to the target RNA and result in sequence-specific degradation of the target RNA through an RNA interference mechanism. Optionally, the siRNA molecules comprise a 3′ hydroxyl group.

A “target molecule” is any compound of interest, including polypeptides, small molecules, ions, large organic molecules (such as various polymers and copolymers), as well as complexes comprising one or more molecular species.

III. Exemplary RNAi Constructs

In certain embodiments, the disclosure provides RNAi constructs containing one or more modifications such that the RNAi constructs have improved cellular uptake. RNAi constructs disclosed herein may have desirable pharmacokinetic properties, such as a reduced clearance rate and a longer serum half-life. The modifications may be selected so as to increase serum stability and/or cellular uptake. The modifications may be selected so as to increase the noncovalent association of the RNAi constructs with proteins. For example, modifications that decrease the overall negative charge and/or increase the hydrophobicity of an RNAi construct will tend to increase noncovalent association with proteins.

RNAi constructs may be designed to contain a nucleotide sequence that hybridizes under physiologic conditions of the cell to the nucleotide sequence of at least a portion of the mRNA transcript for the gene to be inhibited (i.e., the “target” gene) and is sufficient for silencing the target gene. The RNAi construct need only be sufficiently similar to natural RNA that it has the ability to mediate RNAi. Thus, sequence variations that might be expected due to genetic mutation, strain polymorphism or evolutionary divergence may be tolerated. Optionally, the number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

In certain embodiments, a double-stranded RNAi construct may comprise mismatched base pairs. In certain embodiments, the RNAi nucleic acid has a Tm lower than the Tm of a double-stranded nucleic acid comprising the same RNA antisense strand complemented by a perfectly matched sense strand. The Tm comparison is based on Tms of the nucleic acids under the same ionic strength and preferably, physiological ionic strength (e.g., equivalent to about 150 mM NaCl). The Tm may be lower by 1° C., 2 C., 3° C., 4° C., 5° C., 10° C., 15° C., or 20° C. Examples of physiological salt solutions include Frog Ringer, Krebs, Tyrode, Ringer-Locke, De Jalen, and Artificial cerebral spinal fluid. (See Glaxo Wellcome Pharmacology Guide). Tm may be calculated by the accepted formulas. For example:
Tm=81.5+16.6×Log 10[Na⁺]+0.41(% GC)−600/size Formula for Tm Calculation
[Na+] is set to 100 mM, for [Na⁺] up to 0.4M.

Example: 5′-ATGCATGCATGCATGCATG3′ 20 mer; GC=50%; AT=50%
Tm=81.5+16.6×Log 10[0.100]+0.41×50−600/20
Tm=81.5−16.6+0.41×50−600/20=55.4° C.
Tm for same oligo using 2(A+T)+4(C+G)=60° C.
(Tm For Oligos shorter than 25 bp=2(A+T)+4(C+G))

Mismatches are known in the art to destabilize the duplex of a double-stranded nucleic acid. Mismatches can be detected by a variety of methods including measuring the susceptibility of the duplex to certain chemical modifications (e.g., requiring flexibility and space of each strand) (see, e.g., John and Weeks, Biochemistry (2002) 41:6866-74). Mismatch in a DNA:RNA hybrid duplex can also be determined by using RNaseA analysis, because RNases A degrades RNA at sites of single base pair mismatches in a DNA:RNA hybrid.

While not wishing to be bound by any particular theory, mismatches in a double-stranded RNAi construct may induce dissociation of the duplex so as to resemble two single-stranded polynucleotides, which do not induce non-specific effect as a double-stranded RNAi construct may do.

In certain embodiments, a double-stranded RNAi construct may be a DNA:RNA construct, an RNA:RNA construct or an XNA:RNA construct. A DNA:RNA construct is one in which the sense strand comprises at least 50% deoxyribonucleic acids, or modifications thereof, while the antisense strand comprises at least 50% ribonucleic acids, or modifications thereof. An RNA:RNA construct is one in which both the sense and antisense strands comprise at least 50% ribonucleic acids, or modifications thereof. As described herein, a double-stranded nucleic acid may be formed from a single nucleic acid strand that adopts a hairpin or other folding conformation such that two portions of the single nucleic acid hybridize and form the sense and antisense strands of a double helix. Both DNA:RNA and RNA:RNA constructs can be formulated in a hairpin or other folded single strand forms. The terms deoxyribonucleic acid and ribonucleic acid are chemical names that imply a particular ribose-based backbone. Certain modified nucleic acids, such as peptide nucleic acids (PNAs) do not have a ribose-based background. Other modified nucleic acids are modified on the 2′ position of the ribose, such that classification as an RNA or DNA is not possible. These types of nucleic acids may be referred to as “XNAs”. In certain embodiments, the disclosure is intended to encompass XNA:RNA constructs, where “XNA” indicates that the predominant nucleotides of the sense strand are ones that do not have DNA or RNA backbones. For example, if the sense strand comprises greater than 50% peptide nucleic acids, or modifications thereof, the double-stranded construct may be referred to as a PNA:RNA construct. It is understood that a mixed polymer of DNA, RNA and XNA can be conceived that is, according to the above definitions, not termed DNA, RNA or XNA (e.g., a nucleic acid comprising 30% DNA, 30% RNA and 40% XNA). Such mixed nucleic acid strands are explicitly encompassed in the term “nucleic acid”, and it is understood that a nucleic acid may comprise 0, 5, 10, 20, 25, 30, 40 or 50% or more DNA; 0, 5, 10, 20, 25, 30, 40, or 50% or more RNA; and 0, 5, 10, 20, 25, 30, 40 or 50% or more XNA. A nucleic acid comprising 50% RNA and 50% DNA or XNA shall be considered an RNA strand, and a nucleic acid comprising 50% DNA and 50% XNA shall be considered a DNA strand.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro.

One or two strands of an RNAi construct will include modifications to the phosphate-sugar backbone and/or the nucleoside. In general, the sense strand is subject to few constraints in the amount and type of modifications that may be introduced. The sense strand should retain the ability to hybridize with the antisense strand, and, in the case of longer nucleic acids, should not interfere with the activity of RNAses, such as Dicer, that participate in cleaving longer double-stranded constructs to yield smaller, active siRNAs. The antisense strand should retain the ability to hybridize with both the sense strand and the target transcript, and the ability to form an RNAi induced silencing complex (RISC). In certain preferred embodiments, the sense strand comprises entirely modified nucleic acids, while the antisense strand is RNA comprising no more than 0%, 10%, 20%, 30%, 40% or 50% modified nucleic acids. In certain embodiments, the RNAi construct is a RNA(sense):RNA(antisense) construct wherein the RNA(sense) portion comprises one or more modifications. In certain embodiments, the RNAi construct is a DNA(sense):RNA(antisense) construct wherein the DNA(sense) portion comprises one or more modification. Optionally, the RNA(antisense) portion also comprises one or more modification. Modifications will be useful for improving uptake of the construct and/or conferring a longer serum half-life. Additionally, the same modifications, or additional modifications, may confer additional benefits, e.g., reduced susceptibility to cellular nucleases, improved bioavailability, improved formulation characteristics, and/or changed pharmacokinetic properties.

In certain embodiments, the invention provides for modifications of the polynucleotide strands of the RNAi construct which comprise one or more aptamers. An aptamer is a nucleic acid that interacts with a target of interest to form an aptamer:target complex. The aptamer may occur on either the sense or antisense strand and may occur at either the 3′ or 5′ end of either strand, although it is expected that aptamers positioned at the 5′ end of the sense strand will tend to have fewer detrimental effects on the RNAi activity of the construct. Incorporation or attachment of the aptamer to the sense or antisense strand allows each component to retain its activity; that is, the aptamer component retains the ability to interact with a specific target, and the sense and/or antisense strands retain their ability to inhibit target gene expression by an RNAi mechanism. On incorporation or attachment of the aptamer to the sense or antisense strand, these components may also retain certain structural elements, such as secondary or tertiary structure, which were possessed prior to incorporation or attachment. While typically an aptamer will be incorporated into a linear nucleic acid backbone of the RNAi construct, an aptamer may be attached to nucleic acids of an RNAi construct through an alternative bonding arrangement. For example, the aptamer may be attached to a reactive group of a nucleotide to create a branched backbone nucleic acid, where one branch corresponds to the aptamer. In some embodiments, the aptamer may be selected from a plurality of aptamers (e.g. from a nucleic acid library) which may have been screened and/or optimized to impute a beneficial property onto the system, such as binding to a particular target. The aptamers of the present invention may be chemically synthesized and developed in vitro through the SELEX process. The aptamer may be chosen to preferentially interact with and/or bind to a target. Suitable examples of such targets include molecules such as small organic molecules, nucleotides, polynucleotides, peptides, polypeptides, and proteins. Other targets include larger structures such as organelles, viruses, and cells. Examples of suitable proteins include extracellular proteins, membrane proteins, cell surface proteins, or serum proteins (e.g. an albumin such as human serum albumin). Such target molecules may be internalized by a cell. Interaction of the aptamer with the target molecule (e.g. peptide, protein, etc.) may improve bioavailability and/or cellular uptake of the aptamer and/or polynucleotide. The aptamer and/or polynucleotide may be internalized by a cell, and binding of the aptamer to a target molecule, such as a peptide, polypeptide, or protein, may facilitate internalization of the polynucleotide into the cell. Modifications that may be made to the polynucleotides of the instant invention may also be made to one or more aptamers.

Aptamers for use in various embodiments of the invention include any nucleic acid sequence that interacts with a target or target molecule. The interaction may involve direct or indirect binding, and will preferably be a specific interaction. An aptamer may be a naturally occurring nucleic acid sequence or a nucleic acid sequence that is generated in vitro. Many sequences generated in vitro will, by chance or otherwise, also be found in nature. While the technology is available to generate aptamers of any type of nucleic acid, including single- and double-stranded nucleic acids, DNAs, RNAs and polymers comprising nucleic acid analogs, many embodiments described herein preferably employ a single-stranded RNA aptamer.

In certain preferred embodiments, the aptamer is any RNA sequence that specifically interacts with a target molecule. RNA aptamer sequences are known for many target molecules, and it is possible to generate RNA sequences, known as aptamers, that bind small molecules with high affinity and specificity (Wilson, D.; Szostak, J.Annu.Rev.Biochem.1999, 68, 611-647). For example, methods are well established for generating aptamers that bind to antibiotics. See, e.g., Wallace S T, Schroeder R “In vitro selection and characterization of RNAs with high affinity to antibiotics” RNA-Ligand Interactions, Part B; Methods In Enzymology 318:214-229, 2000. Such techniques have been used, for example to select an aptamer to Kanamycin B (Kwon M, Chun S M, Jeong S, Yu J (2001) “In vitro selection of RNA against kanamycin B,” Molecules and Cells 11: (3) 303-311).

Aptamer sequences also can be generated according to methods known to one of skill in the art, including, for example, the SELEX method described in the following references: U.S. Pat. Nos. 5,475,096; 5,595,877; 5,670,637; 5,696,249; 5,773,598; 5,817,785. The SELEX method is summarized below. A pool of diverse DNA molecules is chemically synthesized, such that a randomized or otherwise variable sequence is flanked by constant sequences. A DNA molecule having a variable sequence flanked by constant sequences may be generated, for example, by programming a DNA synthesizer to add discrete nucleotides (e.g. an A, T, G or C) to the growing polynucleotides during synthesis of constant regions and to add mixtures of nucleotides (e.g. an A/T mixture, an A/T/G mixture or an A/T/G/C mixture) to the growing polynucleotides during synthesis of the variable region. When an A/T mixture is added to growing polynucleotides, the result will be a mixture of polynucleotides, some having an A at the newly synthesized position, and some having a T at the newly synthesized position. One of the constant regions generally comprises an RNA polymerase promoter (e.g. a T7 RNA polymerase promoter) positioned to allow transcription of the variable sequence and, optionally, portions of or all of one or both of the flanking constant sequences. The RNA molecules are then partitioned according to a desired characteristic, such as the ability to bind to a target molecule. For example, a target molecule may be affixed to a resin and poured into a chromatography column. The RNA molecules are then passed over the column. Those that do not bind are discarded. RNAs that do bind the target molecule column may be eluted (e.g. with excess of the target molecule, or a guanidinium-HCl or urea solution). These binding RNAs are then converted back into DNA using reverse transcriptase, amplified by polymerase chain reaction (which may involve the use of primers that restore the RNA polymerase promoter, if necessary). The cycle may then be repeated progressively enriching for aptamers that have a potent affinity for the target molecule. In instances where it is desirable to obtain an aptamer that binds to a target molecule but does not bind to another compound (such as a structurally similar precursor molecule), additional selections may be performed to remove those aptamers that bind to the non-target molecule. For example, a column of aptamers bound to the target molecule may be flushed with the non-target molecule to remove aptamers with significant interaction with the non-target molecule. These methods are adaptable for generating single stranded or double stranded aptamers. (Thiesen H-J, Bach C. (1990) Nucleic Acids Res. 18:3203-09; Ellington A D, Szostak J W (1992) Nature 355:850-52). Using techniques such as SELEX, one of skill in the art can generate an aptamer sequence capable of interacting with a target molecule, and the degree of specificity of binding (i.e. lack of binding to other compounds) can also be selected.

Many natural sequences with specific binding properties are also known, and nucleic acids encoding such sequences may be used as aptamer coding sequences of the invention. For example, if the target molecule is coenzyme B12, the 5′untranslated region of the E. coli btuB gene may be used as an aptamer (Nahvi et al. 2002, Chemistry & Biology 9:1043-49). Other naturally occurring nucleic acids that bind possible target molecules are also known (see, for example, Miranda-Rios et al. 2001, Proc. Natl. Acad. Sci. USA 98:9736-41).

Aptamers suitable for use in the methods described herein may be selected empirically. A set of candidate aptamers may be screened by testing the candidates for binding to target. The target binding activity may be situated entirely within an aptamer portion that is non-overlapping with the antisense and sense portions of the RNAi construct that mediate inhibition of gene expression. The target binding activity may also be situated partially or, in unusual instances, entirely within the sense and/or antisense portions of the RNAi construct. In other words, in one approach, an aptamer is selected for target binding without reference to the RNAi constructs that it may be combined with. In such instances, it is expected that the aptamer will retain target binding when it is incorporated into an RNAi construct, and that the other portions of the RNAi construct will show little or no participation in target binding. In such a case, the library of aptamers for screening may be essentially any library containing varied nucleic acid sequences of appropriate length. In other instances, it may it may be desirable to construct an RNAi construct in which a portion of the target binding (aptamer) activity is situated within portions of the RNAi construct that may participate in suppression of gene expression. This may be accomplished by generating an aptamer screening library that contains, as a constant, or relatively constant, portion, the sense or antisense portions of an RNAi construct, or the entire double-stranded RNAi construct (particularly in the case of hairpin RNAi constructs). The affinity and/or specificity of the interaction between an aptamer or aptamer-containing nucleic acid and the target molecule may be measured, and such information may be useful for selecting or describing aptamers that are appropriate for a particular task.

As described above, it is possible to generate aptamers that vary in their binding affinities for the target molecule. The importance of using an aptamer with a high or low affinity for the target molecule will depend on the nature of the intended use for the construct and as discussed above, the affinity will often be of secondary importance to other properties, such as the ability of the aptamer-containing RNAi construct to inhibit gene expression. The term low affinity is used herein to refer to aptamers having a dissociation constant (K_{D}) of 10⁻⁴M or greater. The term moderate affinity is used herein to refer to aptamers having a K_{D} of between 10⁻⁶M and 10⁻⁴M. The term high affinity is used herein to refer to aptamers having a K_{D} of less than 10⁻⁶M. Where the target protein is highly abundant, as in the case of serum albumin, it is expected that even low or moderate affinity aptamers will be adequate. Where the target protein is a rare protein, such as a low-abundance, cell type-specific receptor, a higher affinity aptamer may be effective. A tandem series of aptamers may also be employed. Tandem aptamers may be targeted at the same target, in which case it is generally expected that tandem aptamers will have a lower off-rate than a single aptamer, or targeted to distinct targets, which may increase specific delivery to, for example, cells having both targets.

As described above, it is possible to generate aptamers having a range of different specificities with respect to the target molecule. Specificity, as the term is used herein, is defined relative to a particular non-target molecule. Specificity is herein defined as the ratio of the K_{D} of the aptamer for binding the target molecule to the K_{D} of the aptamer for binding a particular non-target molecule. For example, if the aptamer has a K_{D} of 10⁻⁶M for the target molecule and 10⁻⁵M for the non-target molecule, the specificity is 10 (10⁻⁶/10⁻⁵). The importance of using an aptamer with a high or low specificity for the target molecule relative to a particular non-target molecule will depend on the nature of the intended use.

As one of skill in the art will recognize upon reviewing this disclosure, the methods of the invention can be used with a wide variety of target molecules. One desirable category of targets is proteins that facilitate internalization of bound substances into the cell. When a target molecule is not cell permeable, the target molecule can be applied to the host cell with an adjuvant, carrier, or other material that promotes cell permeabilization. Suitable agents include lipids, liposomes, polymers, and the like, including polycyclodextrin compounds.

One of skill in the art will also readily appreciate that modifications to the nucleotides of the RNAi constructs discussed herein are applicable to the aptamers of the present invention. For example phosphodiester linkages of one or more aptamers may be modified to include one or more nitrogen or sulfur heteroatoms; the aptamers may be modified to include phosphorothioate modifications. In addition to modifications to the aptamer sugar-phosphate backbone, if present, modifications may also be made to the nucleoside portion of the aptamers to include, for example, non-natural bases. Any modification to nucleotides that is known in the art is also applicable to the aptamers of the present invention. Additionally, the aptamers may be composed of primarily of RNA, DNA, XNA, or a mixture of any of these.

Furthermore, in view of this specification, many examples of modifications that decrease the negative charge and/or increase the hydrophobicity of the RNAi construct will be apparent. For example, the phosphodiester linkages of natural RNA may be modified to include at least one of an nitrogen or sulfur heteroatom. Modifications may be assessed for toxic effects on cells in vitro prior to use in vivo. For example, greater than 50% phosphorothioate modifications in the sense or antisense strands may have toxic effects. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis. Hydrophobicity may be assessed by analysis of log P. “Log P” refers to the logarithm of P (Partition Coefficient). P is a measure of how well a substance partitions between a lipid (oil) and water. P itself is a constant. It is defined as the ratio of concentration of compound in aqueous phase to the concentration of compound in an immiscible solvent, as the neutral molecule.
Partition Coefficient, P=[Organic]/[Aqueous] where []=concentration
Log P=log₁₀(Partition Coefficient)=log₁₀ P

In practice, the Log P value will vary according to the conditions under which it is measured and the choice of partitioning solvent. A Log P value of 1 means that the concentration of the compound is ten times greater in the organic phase than in the aqueous phase. The increase in a log P value of 1 indicates a ten fold increase in the concentration of the compound in the organic phase as compared to the aqueous phase. Thus, a compound with a log P value of 3 is 10 times more soluble in water than a compound with a log P value of 4 and a compound with a log P value of 3 is 100 times more soluble in water than a compound with a log P value of 5. In general, compounds having log P values between 7-10 are considered low solubility compounds.

In certain embodiments, the RNAi construct comprising the one or more modifications has a log P value at least 1 log P unit less than the log P value of an otherwise identical unmodified RNAi construct, and preferably at least 2, 3 or even 4 log P unit less than the log P value of an otherwise identical unmodified RNAi construct.

Charge may be determined by measuring the isoelectric point (pI) of the RNAi construct, which may be done, for example, by performing an isoelectric focusing analysis. In certain embodiments, the RNAi construct comprising the one or more modifications has an isoelectric pH (pI) that is at least 0.25 units higher than the otherwise identical unmodified RNAi construct, and preferably at least 0.5, 1 or even 2 units higher than the otherwise identical unmodified RNAi construct.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). Additional modified nucleotides are as follows (this list contains forms that are modified on either the backbone or the nucleoside or both, and is not intended to be all-inclusive): 2′-O-Methyl-2-aminoadenosine; 2′-O-Methyl-5-methyluridine; 2′-O-Methyladenosine; 2′-O-Methylcytidine; 2′-O-Methylguanosine; 2′-O-Methyluridine; 2-Amino-2′-deoxyadenosine; 2-Aminoadenosine; 2-Aminopurine-2′-deoxyriboside; 4-Thiothymidine; 4-Thiouridine; 5-Methyl-2′-deoxycytidine; 5-Methylcytidine; 5-Methyluridine; 5-Propynyl-2′-deoxycytidine; 5-Propynyl-2′-deoxyuridine; N1-Methyladenosine; N1-Methylguanosine; N2-Methyl-2′-deoxyguanosine; N6-Methyl-2′-deoxyadenosine; N6-Methyladenosine; O6-Methyl-2′-deoxyguanosine; and O6-Methylguanosine. A variety of chemical synthetic approaches are available for the conjugation of additional moieties to nucleic acids. For example, one may synthesize nucleic acid-lipid, nucleic acid-sugar conjugates (see, e.g., Anno et al. Nucleosides Nucleotides Nucleic Acids. May-August 2003;22(5-8):1451-3; Watal et al. Nucleic Acids Symp Ser. 2000;(44):179-80), nucleic acid-sterol conjugates or conjugates of other relatively fat soluble hydrophobic moieties such as vitamin E, dodecanol, arachidonic acid, folic acid and retinoic acid (see, e.g., Spiller et al., Blood. Jun. 15, 1998;91(12):4738-46; Bioconjug Chem. March-April1998;9(2):283-91; Lorenz et al. Bioorg Med Chem Lett. Oct. 4, 2004; 14(19):4975-7; Soutschek et al. Nature. Nov. 11, 2004;432(7014):173-8). See also the review of nucleic acid conjugates in Manoharan Antisense Nucleic Acid Drug Dev. April 2002;12(2):103-28. The modifications above are also applicable to the aptamers of the present invention.

The double-stranded structure may be formed by a single self-complementary nucleic acid strand or two complementary nucleic acid strands. Duplex formation may be initiated either inside or outside the cell. The RNAi construct may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Given the greater uptake of the modified RNAi nucleic acids disclosed herein, it is understood that lower dosing may be employed than is generally used with traditional RNAi constructs. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids include an antisense RNA strand that is around 19-30 nucleotides in length, and even more preferably 21-23 nucleotides in length, e.g., corresponding in length to the fragments generated by nuclease “dicing” of long double-stranded RNAs. siRNAs may include a sense strand that is RNA, DNA or XNA. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA antisense molecules comprise a 3′ hydroxyl group. Optionally, the sense strand comprises at least 50%, 60%, 70%, 80%, 90% or 100% modified nucleic acids, while the antisense strand is unmodified RNA. Optionally, the sense strand comprises 100% modified nucleic acids (e.g. DNA or RNA with a phosphorothioate modification at every possible position) while the antisense strand is an RNA strand comprising no modified nucleic acids or no more than 10%, 20%, 30%, 40% or 50% modified RNA nucleic acids.

The siRNA molecules of the present invention can be obtained using a number of techniques known to those of skill in the art. For example, the siRNA can be chemically synthesized or recombinantly produced using methods known in the art. For example, short sense and antisense RNA, DNA or XNA oligomers can be synthesized and annealed to form double-stranded structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be introduced into cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides. In this embodiment, modifications should be selected so as to not interfere with the activity of the RNAse.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA antisense strand is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA:RNA or DNA:RNA hybrid or XNA:RNA:. In certain embodiments, the RNAi construct is at least 25, 50, 100, 200, 300 or 400 bases. In certain embodiments, the RNAi construct is 400-800 bases in length. The double-stranded nucleic acids are digested intracellularly, e.g., to produce siRNA sequences in the cell. However, use of long double-stranded nucleic acids in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, an RNAi construct is in the form of a hairpin structure. The hairpin can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell. A hairpin may be chemically synthesized such that a sense strand comprises RNA, DNA or XNA, while the antisense strand comprises RNA. In such an embodiment, the single strand portion connecting the sense and antisense portions, sometimes referred to as the loop portion, should be designed so as to be cleavable by nucleases in vivo, and any duplex portion should be susceptible to processing by nucleases such as Dicer.

In certain embodiments that comprise one or more modifications to the RNAi construct which comprise one or more aptamers, such aptamers are compatible with the hairpin structure of the RNAi construct. The aptamers may be associated with either the sense or antisense portion of the duplex, or double-stranded, portion of the hairpin. The aptamers may also be associated with the loop portion of the hairpin.

IV. Exemplary Formulations

The RNAi constructs of the invention may also be admixed, encapsulated, conjugated or otherwise associated with other molecules, molecule structures or mixtures of compounds, as for example, liposomes, polymers, receptor targeted molecules, oral, rectal, topical or other formulations, for assisting in uptake, distribution and/or absorption. The subject RNAi constructs can be provided in formulations also including penetration enhancers, carrier compounds and/or transfection agents.

In certain embodiments, the increased association of the RNAi constructs disclosed herein may be used to generate pre-associated mixtures comprising an RNAi construct and a protein. For example, a composition for delivery to a subject may comprise one or more serum proteins, such as albumin (preferably matched to the species for deliver, e.g. human serum albumin for delivery to a human) and an RNAi construct. Thus, a significant percentage of the RNAi construct will be associated with protein at the time of delivery to the subject. A protein may be selected to be appropriate for the delivery mode. Serum proteins are particularly suitable for delivery to any portion of the body perfused with blood, and particularly for intravenous administration. Mucoid proteins or proteoglycans may be desirable for administration to a mucosal surface, such as the airways, rectum, eye or genitalia.

A protein may be selected for targeting the RNAi construct to a particular tissue or cell type. For example, a transferrin protein may be used to target the RNAi construct to cells of a neoplasm (“neoplastic cells”). As another example, a protein with one or more galactose moieties may be used to target the RNAi construct to hepatocytes. An RNAi construct may be pre-mixed with an antibody that has affinity for a targeted cell or tissue type. Methods for generating targeting antibodies are well-known in the art. An antibody may be, for example, a monoclonal or polyclonal antibody, a polypeptide comprising a single chain antibody, an Fv fragment, an Fc fragment (e.g., for targeting to Fc binding cells), a chimeric or humanized antibody, a fully human antibody, any type of antibody, such as an IgG, IgM, IgE or IgD or a portion thereof. Additional examples of targeting polypeptides are listed in the Table below.

| Ligand | Receptor | Cell type |
|---|---|---|
| apolipoproteins | LDL | liver hepatocytes, |
| | | vascular endothelial cells |
| insulin | insulin | |
| | receptor | |
| transferrin | transferrin | endothelial cells |
| | receptor | |
| galactose | asialoglyco- | liver hepatocytes |
| | protein | |
| | receptor | |
| Mac-1 | L selectin | neutrophils, leukocytes |
| VEGF | Flk-1, 2 | tumor epithelial cells |
| basic FGF | FGF receptor | tumor epithelial cells |
| EGF | EGF receptor | epithelial cells |
| VCAM-1 | a₄b₁ integrin | vascular endothelial cells |
| ICAM-1 | a_{L}b₂ integrin | vascular endothelial cells |
| PECAM-1/CD31 | aᵥb₃ integrin | vascular endothelial cells, |
| | | activated platelets |
| osteopontin | aᵥb₁ integrin | endothelial cells and |
| | aᵥb₅ integrin | smooth muscle cells in |
| | | atherosclerotic plaques |
| RGD sequences | aᵥb₃ integrin | tumor endothelial cells, |
| | | vascular smooth muscle |
| | | cells |
| HIV GP 120/41 or GP120 | CD4 | CD4 + lymphocytes |

A polypeptide may also be an internalizing polypeptide selected to specifically facilitate uptake into cells. In one embodiment, the internalizing peptide is derived from the Drosophila antepennepedia protein, or homologs thereof. The 60 amino acid long homeodomain of the homeo-protein antepennepedia has been demonstrated to translocate through biological membranes and can facilitate the translocation of heterologous polypeptides to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193. Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8). Peptides or analogs that include a sequence present in the highly basic region, such as CFITKALGISYGRKKRRQRRRPPQGS, are conjugated to the polymer to aid in internalization and targeting those complexes to the intracellular milleau. Another exemplary transcellular polypeptide can be generated to include a sufficient portion of mastoparan (T. Higashijima et al., (1990) J. Biol. Chem. 265:14176) to increase the transmembrane transport of the RNAi complexes.

Other suitable internalizing peptides can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefor serve as an internalizing peptide for the subject transcellular polypeptides. Preferred growth factor-derived internalizing peptides include EGF (epidermal growth factor)-derived peptides, such as CMHIESLDSYTC and CMYIEALDKYAC; TGF-beta (transforming growth factor beta )-derived peptides; peptides derived from PDGF (platelet-derived growth factor) or PDGF-2; peptides derived from IGF-I (insulin-like growth factor) or IGF-II; and FGF (fibroblast growth factor)-derived peptides.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Aptamers of the present invention may be selected and/or optimized for interaction (e.g. binding) with the internalizing peptides discussed above. Such an interaction may facilitate cellular uptake of the aptamer and/or RNAi construct.

A polypeptide may also be a fusion protein, comprising a first domain that is selected or designed for interaction with the RNAi construct and a second domain that is selected or designed for targeting, internalization or other desired functionality.

An RNAi construct may be pre-mixed with a plurality of polypeptide species, optionally of several different types (e.g. a serum protein and a targeting protein). Additional substances may be included as well, such as those described below.

Representative United States patents that teach the preparation of uptake, distribution and/or absorption assisting formulations which can be adapted for delivery of RNAi constructs include, but are not limited to, U.S. Pat. Nos. 5,108,921; 5,354,844; 5,416,016; 5,459,127; 5,521,291; 5,1543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The RNAi constructs of the invention also encompass any pharmaceutically acceptable salts, esters or salts of such esters, or any other compound which, upon administration to an animal including a human, is capable of providing (directly or indirectly) the biologically active metabolite or residue thereof. Accordingly, for example, the disclosure is also drawn to RNAi constructs and pharmaceutically acceptable salts of the siRNAs, pharmaceutically acceptable salts of such RNAi constructs, and other bioequivalents.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,NI-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66,1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

For siRNA oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalene disulfonic acid, polygalacturonic acid, and the like; and (d) salts formed from elemental anions such as chlorine, bromine, and iodine.

Another aspect of the invention provides aerosols for the delivery of RNAi constructs to the respiratory tract. The respiratory tract includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli which then lead to the ultimate respiratory zone, the alveoli, or deep lung.

Herein, administration by inhalation may be oral and/or nasal. Examples of pharmaceutical devices for aerosol delivery include metered dose inhalers (MDIs), dry powder inhalers (DPIs), and air-jet nebulizers. Exemplary nucleic acid delivery systems by inhalation which can be readily adapted for delivery of the subject RNAi constructs are described in, for example, U.S. Pat. Nos. 5,756,353; 5,858,784; and PCT applications WO98/31346; WO98/10796; WO00/27359; WO01/54664; WO02/060412. Other aerosol formulations that may be used for delivering the double-stranded RNAs are described in U.S. Pat. Nos. 6,294,153; 6,344,194; 6,071,497, and PCT applications WO02/066078; WO02/053190; WO01/60420; WO00/66206. Further, methods for delivering RNAi constructs can be adapted from those used in delivering other oligonucleotides (e.g., an antisense oligonucleotide) by inhalation, such as described in Templin et al., Antisense Nucleic Acid Drug Dev, 2000, 10:359-68; Sandrasagra et al., Expert Opin Biol Ther, 2001, 1:979-83; Sandrasagra et al., Antisense Nucleic Acid Drug Dev, 2002, 12:177-81.

The human lungs can remove or rapidly degrade hydrolytically cleavable deposited aerosols over periods ranging from minutes to hours. In the upper airways, ciliated epithelia contribute to the “mucociliary excalator” by which particles are swept from the airways toward the mouth. Pavia, D., “LungMucociliary Clearance,” in Aerosols and the Lung: Clinical and Experimental Aspects, Clarke, S. W. and Pavia, D., Eds., Butterworths, London, 1984. In the deep lungs, alveolar macrophages are capable of phagocytosing particles soon after their deposition. Warheit et al. Microscopy Res. Tech., 26: 412-422 (1993); and Brain, J. D., “Physiology and Pathophysiology of Pulmonary Macrophages,” in The Reticuloendothelial System, S. M. Reichard and J. Filkins, Eds., Plenum, N.Y., pp. 315-327, 1985. The deep lung, or alveoli, are the primary target of inhaled therapeutic aerosols for systemic delivery of RNAi constructs.

In preferred embodiments, particularly where systemic dosing with the RNAi construct is desired, the aerosoled RNAi constructs are formulated as microparticles. Microparticles having a diameter of between 0.5 and ten microns can penetrate the lungs, passing through most of the natural barriers. A diameter of less than ten microns is required to bypass the throat; a diameter of 0.5 microns or greater is required to avoid being exhaled.

Another aspect of the invention relates to coated medical devices. For instance, in certain embodiments, the subject invention provides a medical device having a coating adhered to at least one surface, wherein the coating includes the subject polymer matrix and an RNAi construct containing modifications as disclosed herein. Optionally the coating further comprises protein noncovalently associated with the RNAi construct (or selected to interact with the RNAi construct upon release from the coating). Such coatings can be applied to surgical implements such as screws, plates, washers, sutures, prosthesis anchors, tacks, staples, electrical leads, valves, membranes. The devices can be catheters, implantable vascular access ports, blood storage bags, blood tubing, central venous catheters, arterial catheters, vascular grafts, intraaortic balloon pumps, heart valves, cardiovascular sutures, artificial hearts, a pacemaker, ventricular assist pumps, extracorporeal devices, blood filters, hemodialysis units, hemoperfasion units, plasmapheresis units, and filters adapted for deployment in a blood vessel.

In some embodiments according to the present invention, monomers for forming a polymer are combined with an RNAi construct and are mixed to make a homogeneous dispersion of the RNAi construct in the monomer solution. The dispersion is then applied to a stent or other device according to a conventional coating process, after which the crosslinking process is initiated by a conventional initiator, such as UV light. In other embodiments according to the present invention, a polymer composition is combined with an RNAi construct to form a dispersion. The dispersion is then applied to a surface of a medical device and the polymer is cross-linked to form a solid coating. In other embodiments according to the present invention, a polymer and an RNAi construct are combined with a suitable solvent to form a dispersion, which is then applied to a stent in a conventional fashion. The solvent is then removed by a conventional process, such as heat evaporation, with the result that the polymer and RNAi construct (together forming a sustained-release drug delivery system) remain on the stent as a coating. An analogous process may be used where the RNAi construct is dissolved in the polymer composition. Where the RNAi is to be pre-mixed with a protein, solvents are preferably selected so as to preserve the tertiary structure of the protein.

In some embodiments according to the invention, the system comprises a polymer that is relatively rigid. In other embodiments, the system comprises a polymer that is soft and malleable. In still other embodiments, the system includes a polymer that has an adhesive character. Hardness, elasticity, adhesive, and other characteristics of the polymer are widely variable, depending upon the particular final physical form of the system, as discussed in more detail below.

Embodiments of the system according to the present invention take many different forms. In some embodiments, the system consists of the RNAi construct suspended or dispersed in the polymer. In certain other embodiments, the system consists of an RNAi construct and a semi solid or gel polymer, which is adapted to be injected via a syringe into a body. In other embodiments according to the present invention, the system consists of an RNAi construct and a soft flexible polymer, which is adapted to be inserted or implanted into a body by a suitable surgical method. In still further embodiments according to the present invention, the system consists of a hard, solid polymer, which is adapted to be inserted or implanted into a body by a suitable surgical method. In further embodiments, the system comprises a polymer having the RNAi construct suspended or dispersed therein, wherein the RNAi construct and polymer mixture forms a coating on a surgical implement, such as a screw, stent, pacemaker, etc. In particular embodiments according to the present invention, the device consists of a hard, solid polymer, which is shaped in the form of a surgical implement such as a surgical screw, plate, stent, etc., or some part thereof. In other embodiments according to the present invention, the system includes a polymer that is in the form of a suture having the RNAi construct dispersed or suspended therein.

In some embodiments according to the present invention, provided is a medical device comprising a substrate having a surface, such as an exterior surface, and a coating on the exterior surface. The coating comprises a polymer and an RNAi construct dispersed in the polymer, wherein the polymer is permeable to the RNAi construct or biodegrades to release the RNAi construct. Optionally, the coating further comprises a protein that associates with the RNAi construct. In certain embodiments according to the present invention, the device comprises an RNAi construct suspended or dispersed in a suitable polymer, wherein the RNAi construct and polymer are coated onto an entire substrate, e.g., a surgical implement. Such coating may be accomplished by spray coating or dip coating.

In other embodiments according to the present invention, the device comprises an RNAi construct and polymer suspension or dispersion, wherein the polymer is rigid, and forms a constituent part of a device to be inserted or implanted into a body. Optionally, the suspension or dispersion further comprises a polypeptide that non-covalently interacts with the RNAi construct. For instance, in particular embodiments according to the present invention, the device is a surgical screw, stent, pacemaker, etc. coated with the RNAi construct suspended or dispersed in the polymer. In other particular embodiments according to the present invention, the polymer in which the RNAi construct is suspended forms a tip or a head, or part thereof, of a surgical screw. In other embodiments according to the present invention, the polymer in which RNAi construct is suspended or dispersed is coated onto a surgical implement such as surgical tubing (such as colostomy, peritoneal lavage, catheter, and intravenous tubing). In still further embodiments according to the present invention, the device is an intravenous needle having the polymer and RNAi construct coated thereon.

As discussed above, the coating according to the present invention comprises a polymer that is bioerodible or non bioerodible. The choice of bioerodible versus non-bioerodible polymer is made based upon the intended end use of the system or device. In some embodiments according to the present invention, the polymer is advantageously bioerodible. For instance, where the system is a coating on a surgically implantable device, such as a screw, stent, pacemaker, etc., the polymer is advantageously bioerodible. Other embodiments according to the present invention in which the polymer is advantageously bioerodible include devices that are implantable, inhalable, or injectable suspensions or dispersions of RNAi construct in a polymer, wherein the further elements (such as screws or anchors) are not utilized.

In some embodiments according to the present invention wherein the polymer is poorly permeable and bioerodible, the rate of bioerosion of the polymer is advantageously sufficiently slower than the rate of RNAi construct release so that the polymer remains in place for a substantial period of time after the RNAi construct has been released, but is eventually bioeroded and resorbed into the surrounding tissue. For example, where the device is a bioerodible suture comprising the RNAi construct suspended or dispersed in a bioerodible polymer, the rate of bioerosion of the polymer is advantageously slow enough that the RNAi construct is released in a linear manner over a period of about three to about 14 days, but the sutures persist for a period of about three weeks to about six months. Similar devices according to the present invention include surgical staples comprising an RNAi construct suspended or dispersed in a bioerodible polymer.

In other embodiments according to the present invention, the rate of bioerosion of the polymer is advantageously on the same order as the rate of RNAi construct release. For instance, where the system comprises an RNAi construct suspended or dispersed in a polymer that is coated onto a surgical implement, such as an orthopedic screw, a stent, a pacemaker, or a non-bioerodible suture, the polymer advantageously bioerodes at such a rate that the surface area of the RNAi construct that is directly exposed to the surrounding body tissue remains substantially constant over time.

In other embodiments according to the present invention, the polymer vehicle is permeable to water in the surrounding tissue, e.g. in blood plasma. In such cases, water solution may permeate the polymer, thereby contacting the RNAi construct. The rate of dissolution may be governed by a complex set of variables, such as the polymer's permeability, the solubility of the RNAi construct, the pH, ionic strength, and protein composition, etc. of the physiologic fluid.

In some embodiments according to the present invention, the polymer is non-bioerodible. Non bioerodible polymers are especially useful where the system includes a polymer intended to be coated onto, or form a constituent part, of a surgical implement that is adapted to be permanently, or semi permanently, inserted or implanted into a body. Exemplary devices in which the polymer advantageously forms a permanent coating on a surgical implement include an orthopedic screw, a stent, a prosthetic joint, an artificial valve, a permanent suture, a pacemaker, etc.

There are a multiplicity of different stents that may be utilized following percutaneous transluminal coronary angioplasty. Although any number of stents may be utilized in accordance with the present invention, for simplicity, a limited number of stents will be described in exemplary embodiments of the present invention. The skilled artisan will recognize that any number of stents may be utilized in connection with the present invention. In addition, as stated above, other medical devices may be utilized.

A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

The stents of the present invention may be fabricated utilizing any number of methods. For example, the stent may be fabricated from a hollow or formed stainless steel tube that may be machined using lasers, electric discharge milling, chemical etching or other means. The stent is inserted into the body and placed at the desired site in an unexpanded form. In one exemplary embodiment, expansion may be effected in a blood vessel by a balloon catheter, where the final diameter of the stent is a function of the diameter of the balloon catheter used.

It should be appreciated that a stent in accordance with the present invention may be embodied in a shape-memory material, including, for example, an appropriate alloy of nickel and titanium or stainless steel.

Structures formed from stainless steel may be made self-expanding by configuring the stainless steel in a predetermined manner, for example, by twisting it into a braided configuration. In this embodiment after the stent has been formed it may be compressed so as to occupy a space sufficiently small as to permit its insertion in a blood vessel or other tissue by insertion means, wherein the insertion means include a suitable catheter, or flexible rod.

On emerging from the catheter, the stent may be configured to expand into the desired configuration where the expansion is automatic or triggered by a change in pressure, temperature or electrical stimulation.

Regardless of the design of the stent, it is preferable to have the RNAi construct, and protein (where applicable), applied with enough specificity and a sufficient concentration to provide an effective dosage in the lesion area. In this regard, the “reservoir size” in the coating is preferably sized to adequately apply the RNAi construct at the desired location and in the desired amount.

In an alternate exemplary embodiment, the entire inner and outer surface of the stent may be coated with the RNAi construct, and optionally protein, in therapeutic dosage amounts. It is, however, important to note that the coating techniques may vary depending on the RNAi construct and any included protein. Also, the coating techniques may vary depending on the material comprising the stent or other intraluminal medical device.

The intraluminal medical device comprises the sustained release drug delivery coating. The RNAi construct coating may be applied to the stent via a conventional coating process, such as impregnating coating, spray coating and dip coating.

In one embodiment, an intraluminal medical device comprises an elongate radially expandable tubular stent having an interior luminal surface and an opposite exterior surface extending along a longitudinal stent axis. The stent may include a permanent implantable stent, an implantable grafted stent, or a temporary stent, wherein the temporary stent is defined as a stent that is expandable inside a vessel and is thereafter retractable from the vessel. The stent configuration may comprise a coil stent, a memory coil stent, a Nitinol stent, a mesh stent, a scaffold stent, a sleeve stent, a permeable stent, a stent having a temperature sensor, a porous stent, and the like. The stent may be deployed according to conventional methodology, such as by an inflatable balloon catheter, by a self-deployment mechanism (after release from a catheter), or by other appropriate means. The elongate radially expandable tubular stent may be a grafted stent, wherein the grafted stent is a composite device having a stent inside or outside of a graft. The graft may be a vascular graft, such as an ePTFE graft, a biological graft, or a woven graft.

The RNAi construct, and any associated protein, may be incorporated onto or affixed to the stent in a number of ways. In the exemplary embodiment, the RNAi construct is directly incorporated into a polymeric matrix and sprayed onto the outer surface of the stent. The RNAi construct elutes from the polymeric matrix over time and enters the surrounding tissue. The RNAi construct preferably remains on the stent for at least three days up to approximately six months, and more preferably between seven and thirty days.

In certain embodiments, the polymer according to the present invention comprises any biologically tolerated polymer that is permeable to the RNAi construct and while having a permeability such that it is not the principal rate determining factor in the rate of release of the RNAi construct from the polymer.

In some embodiments according to the present invention, the polymer is non-bioerodible. Examples of non-bioerodible polymers useful in the present invention include poly(ethylene-co-vinyl acetate) (EVA), polyvinylalcohol and polyurethanes, such as polycarbonate-based polyurethanes. In other embodiments of the present invention, the polymer is bioerodible. Examples of bioerodible polymers useful in the present invention include polyanhydride, polylactic acid, polyglycolic acid, polyorthoester, polyalkylcyanoacrylate or derivatives and copolymers thereof. The skilled artisan will recognize that the choice of bioerodibility or non-bioerodibility of the polymer depends upon the final physical form of the system, as described in greater detail below. Other exemplary polymers include polysilicone and polymers derived from hyaluronic acid. The skilled artisan will understand that the polymer according to the present invention is prepared under conditions suitable to impart permeability such that it is not the principal rate determining factor in the release of the RNAi construct from the polymer.

Moreover, suitable polymers include naturally occurring (collagen, hyaluronic acid, etc.) or synthetic materials that are biologically compatible with bodily fluids and mammalian tissues, and essentially insoluble in bodily fluids with which the polymer will come in contact. In addition, the suitable polymers essentially prevent interaction between the RNAi construct dispersed/suspended in the polymer and proteinaceous components in the bodily fluid. The use of rapidly dissolving polymers or polymers highly soluble in bodily fluid or which permit interaction between the RNAi construct and endogenous proteinaceous components are to be avoided in certain instances since dissolution of the polymer or interaction with proteinaceous components would affect the constancy of drug release. The selection of polymers may differ where the RNAi construct is pre-associated with protein in the coating.

Other suitable polymers include polypropylene, polyester, polyethylene vinyl acetate (PVA or EVA), polyethylene oxide (PEO), polypropylene oxide, polycarboxylic acids, polyalkylacrylates, cellulose ethers, silicone, poly(d1-lactide-co glycolide), various Eudragrits (for example, NE30D, RS PO and RL PO), polyalkyl-alkyacrylate copolymers, polyester-polyurethane block copolymers, polyether-polyurethane block copolymers, polydioxanone, poly-(β-hydroxybutyrate), polylactic acid (PLA), polycaprolactone, polyglycolic acid, and PEO-PLA copolymers.

The coating of the present invention may be formed by mixing one or more suitable monomers and a suitable RNAi construct, then polymerizing the monomer to form the polymer system. In this way, the RNAi construct, and any associated protein, is dissolved or dispersed in the polymer. In other embodiments, the RNAi construct, and any associated protein, is mixed into a liquid polymer or polymer dispersion and then the polymer is further processed to form the inventive coating. Suitable further processing may include crosslinking with suitable crosslinking RNAi constructs, further polymerization of the liquid polymer or polymer dispersion, copolymerization with a suitable monomer, block copolymerization with suitable polymer blocks, etc. The further processing traps the RNAi construct in the polymer so that the RNAi construct is suspended or dispersed in the polymer vehicle.

Any number of non-erodible polymers may be utilized in conjunction with the RNAi construct. Film-forming polymers that can be used for coatings in this application can be absorbable or non-absorbable and must be biocompatible to minimize irritation to the vessel wall. The polymer may be either biostable or bioabsorbable depending on the desired rate of release or the desired degree of polymer stability, but a bioabsorbable polymer may be preferred since, unlike biostable polymer, it will not be present long after implantation to cause any adverse, chronic local response. Furthermore, bioabsorbable polymers do not present the risk that over extended periods of time there could be an adhesion loss between the stent and coating caused by the stresses of the biological environment that could dislodge the coating and introduce further problems even after the stent is encapsulated in tissue.

Suitable film-forming bioabsorbable polymers that could be used include polymers selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, biomolecules and blends thereof. For the purpose of this invention aliphatic polyesters include homopolymers and copolymers of lactide (which includes lactic acid d-,1- and meso lactide), E-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30(1), page 498, 1989 (e.g. PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein). Polyphosphazenes, co-, ter- and higher order mixed monomer based polymers made from L-lactide, D,L-lactide, lactic acid, glycolide, glycolic acid, para-dioxanone, trimethylene carbonate and E-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein). Polyanhydrides from diacids of the form HOOC—C₆H₄—O—(CH₂)ₘ—O—C₆H₄—COOH where m is an integer in the range of from 2 to 8 and copolymers thereof with aliphatic alpha-omega diacids of up to 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference). Film-forming polymeric biomolecules for the purpose of this invention include naturally occurring materials that may be enzymatically degraded in the human body or are hydrolytically unstable in the human body such as fibrin, fibrinogen, collagen, elastin, and absorbable biocompatable polysaccharides such as chitosan, starch, fatty acids (and esters thereof), glucoso-glycans and hyaluronic acid.

Suitable film-forming biostable polymers with relatively low chronic tissue response, such as polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters could also be used. Other polymers could also be used if they can be dissolved, cured or polymerized on the stent. These include polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile, polyvinyl ketones; polyvinyl aromatics such as polystyrene; polyvinyl esters such as polyvinyl acetate; copolymers of vinyl monomers with each other and olefins, such as etheylene-methyl methacrylate copolymers, acrylonitrile-styrene copolymers, ABS resins and ethylene-vinyl acetate copolymers; polyamides,such as Nylon 66 and polycaprolactam; alkyd resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e. carboxymethyl cellulose and hydoxyalkyl celluloses); and combinations thereof. Polyamides for the purpose of this application would also include polyamides of the form —NH—(CH₂)ₙ—CO— and NH—(CH₂)ₓ—NH—CO—(CH₂)_{y}—CO, wherein n is preferably an integer in from 6 to 13; x is an integer in the range of form 6 to 12; and y is an integer in the range of from 4 to 16. The list provided above is illustrative but not limiting.

The polymers used for coatings can be film-forming polymers that have molecular weight high enough as to not be waxy or tacky. The polymers also should adhere to the stent and should not be so readily deformable after deposition on the stent as to be able to be displaced by hemodynamic stresses. The polymers molecular weight be high enough to provide sufficient toughness so that the polymers will not to be rubbed off during handling or deployment of the stent and must not crack during expansion of the stent. In certain embodiments, the polymer has a melting temperature above 40° C., preferably above about 45° C., more preferably above 50° C. and most preferably above 55° C.

Coating may be formulated by mixing one or more of the therapeutic RNAi constructs with the coating polymers in a coating mixture. The RNAi construct may be present as a liquid, a finely divided solid, or any other appropriate physical form. Optionally, the mixture may include one or more proteins that associate with the RNAi construct. Optionally, the mixture may include one or more additives, e.g., nontoxic auxiliary substances such as diluents, carriers, excipients, stabilizers or the like. Other suitable additives may be formulated with the polymer and RNAi construct. For example, hydrophilic polymers selected from the previously described lists of biocompatible film forming polymers may be added to a biocompatible hydrophobic coating to modify the release profile (or a hydrophobic polymer may be added to a hydrophilic coating to modify the release profile). One example would be adding a hydrophilic polymer selected from the group consisting of polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the therapeutic RNAi constructs.

The thickness of the coating can determine the rate at which the RNAi construct elutes from the matrix. Essentially, the RNAi construct elutes from the matrix by diffusion through the polymer matrix. Polymers are permeable, thereby allowing solids, liquids and gases to escape therefrom. The total thickness of the polymeric matrix is in the range from about one micron to about twenty microns or greater. It is important to note that primer layers and metal surface treatments may be utilized before the polymeric matrix is affixed to the medical device. For example, acid cleaning, alkaline (base) cleaning, salinization and parylene deposition may be used as part of the overall process described.

To further illustrate, a poly(ethylene-co-vinylacetate), polybutylmethacrylate and RNAi construct solution may be incorporated into or onto the stent in a number of ways. For example, the solution may be sprayed onto the stent or the stent may be dipped into the solution. Other methods include spin coating and RF plasma polymerization. In one exemplary embodiment, the solution is sprayed onto the stent and then allowed to dry. In another exemplary embodiment, the solution may be electrically charged to one polarity and the stent electrically changed to the opposite polarity. In this manner, the solution and stent will be attracted to one another. In using this type of spraying process, waste may be reduced and more precise control over the thickness of the coat may be achieved.

In another exemplary embodiment, the RNAi construct may be incorporated into a film-forming polyfluoro copolymer comprising an amount of a first moiety selected from the group consisting of polymerized vinylidenefluoride and polymerized tetrafluoroethylene, and an amount of a second moiety other than the first moiety and which is copolymerized with the first moiety, thereby producing the polyfluoro copolymer, the second moiety being capable of providing toughness or elastomeric properties to the polyfluoro copolymer, wherein the relative amounts of the first moiety and the second moiety are effective to provide the coating and film produced therefrom with properties effective for use in treating implantable medical devices.

In one embodiment according to the present invention, the exterior surface of the expandable tubular stent of the intraluminal medical device of the present invention comprises a coating according to the present invention. The exterior surface of a stent having a coating is the tissue-contacting surface and is biocompatible. The “sustained release RNAi construct delivery system coated surface” s synonymous with “coated surface”, which surface is coated, covered or impregnated with a sustained release RNAi construct delivery system according to the present invention.

In an alternate embodiment, the interior luminal surface or entire surface (i.e. both interior and exterior surfaces) of the elongate radially expandable tubular stent of the intraluminal medical device of the present invention has the coated surface. The interior luminal surface having the inventive sustained release RNAi construct delivery system coating is also the fluid contacting surface, and is biocompatible and blood compatible.

V. Exemplary Uses

In general, RNAi has been validated as an effective technique for manipulating expression of essentially any gene in most organisms, including humans. Accordingly, RNAi constructs and formulations disclosed herein may be used to decrease the expression of essentially any target gene, where such decreased expression is expected to provide a desired result, such as an amelioration of a disease (including causal factors and symptoms) or prevention of a disease in an at-risk individual. One need merely select the desired target gene and design the appropriate RNAi construct according to the guidance provided in this specification and in the art generally. Such constructs may be tested on in vitro cell cultures and tissue cultures prior to administration to a living subject. Constructs may also be tested in organisms closely related to the subject species (e.g., monkey models may be tested prior to use of a construct in humans).

In one aspect, the subject method is used to inhibit, or at least reduce, unwanted growth of cells in vivo, and particularly the growth of transformed cells. In certain embodiments, the subject method utilizes RNAi to selectively inhibit the expression of genes encoding proliferation-regulating proteins. For instance, the subject method can be used to inhibit expression of a gene product that is essential to mitosis in the target cell, and/or which is essential to preventing apoptosis of the target cell. The RNAi constructs of the present invention can be designed to correspond to the coding sequence or other portions of mRNAs encoding the targeted proliferation-regulating protein. When treated with the RNAi construct, the loss-of-expression phenotype which results in the target cell causes the cell to become quiescent or to undergo apoptosis.

In certain embodiments, the subject RNAi constructs are selected to inhibit expression of gene products which stimulate cell growth and mitosis. On class of genes which can be targeted by the method of the present invention are those known as oncogenes. As used herein, the term “oncogene” refers to a gene which stimulates cell growth and, when its level of expression in the cell is reduced, the rate of cell growth is reduced or the cell becomes quiescent. In the context of the present invention, oncogenes include intracellular proteins, as well as extracellular growth factors which may stimulate cell proliferation through autocrine or paracrine function. Examples of human oncogenes against which RNAi constructs can designed include c-myc, c-myb, mdm2, PKA-I (protein kinase A type I), Abl-1, Bcl2, Ras, c-Raf kinase, CDC25 phosphatases, cyclins, cyclin dependent kinases (cdks), telomerase, PDGF/sis, erb-B, fos, jun, mos, and src, to name but a few. In the context of the present invention, oncogenes also include a fusion gene resulted from chromosomal translocation, for example, the Bcr/Abl fusion oncogene.

In certain preferred embodiments, the subject RNAi constructs are selected by their ability to inhibit expression of a gene(s) essential for proliferation of a transformed cell, and particularly of a tumor cell. Such RNAi constructs can be used as part of the treatment or prophylaxis for neoplastic, anaplastic and/or hyperplastic cell growth in vivo, including as part of a treatment of a tumor. The c-myc protein is deregulated in many forms of cancer, resulting in increased expression. Reduction of c-myc RNA levels in vitro results in induction of apoptosis. An siRNA complementary to c-myc can therefore be potentially be used as therapeutic for anti-cancer treatment. Preferably, the subject RNAi constructs can be used in the therapeutic treatment of chronic lymphatic leukemia. Chronic lymphatic leukemia is often caused by a translocation of chromosomes 9 and 12 resulting in a Bcr/Abl fusion product. The resulting fusion protein acts as an oncogene; therefore, specific elimination of Bcr/Abl fusion mRNA may result in cell death in the leukemia cells. Indeed, transfection of siRNA molecules specific for the Bcr/Abl fusion mRNA into cultured leukemic cells, not only reduced the fusion mRNA and corresponding oncoprotein, but also induced apoptosis of these cells (see, for example, Wilda et al., Oncogene, 2002, 21:5716-5724).

In other embodiments, the subject RNAi constructs are selected by their ability to inhibit expression of a gene(s) essential for activation of lymphocytes, e.g., proliferation of B-cells or T-cells, and particularly of antigen-mediated activation of lymphocytes. Such RNAi constructs can be used as immunosuppressant agents, e.g., as part of the treatment or prophylaxis for immune-mediated inflammatory disorders.

In certain embodiments, the methods described herein can be employed for the treatment of autoimmune disorders. For example, the subject RNAi constructs are selected for their ability to inhibit expression of a gene(s) which encode or regulate the expression of cytokines. Accordingly, constructs that cause inhibited or decreased expression of cytokines such as THFα, IL-1α, IL-6 or IL-12, or a combination thereof, can be used as part of a treatment or prophylaxis for rheumatoid arthritis. Similarly, constructs that cause inhibited or decreased expression of cytokines involved in inflammation can be used in the treatment or prophylaxis of inflammation and inflammation-related diseases, such as multiple sclerosis.

In other embodiments, the subject RNAi constructs are selected for their ability to inhibit expression of a gene(s) implicated in the onset or progression of diabetes. For example, experimental diabetes mellitus was found to be related to an increase in expression of p21WAF1/CIP1 (p21), and TGF-beta 1 has been implicated in glomerular hypertrophy (see, for example, Al-Douahji, et al. Kidney Int. 56:1691-1699). Accordingly, constructs that cause inhibited or decreased expression of these proteins can be used in the treatment or prophylaxis of diabetes.

In other embodiments, the subject RNAi constructs are selected for their ability to inhibit expression of ICAM-1 (intracellular adhesion molecule). An antisense nucleic acid that inhibits expression of ICAM-1 is being developed by Isis pharmaceutics for psoriasis. Additionally, an antisense nucleic acid against the ICAM-1 gene is suggested for preventing acute renal failure and reperfusion injury and for prolonging renal isograft survival (see, for example, Haller et al. (1996) Kidney Int. 50:473-80; Dragun et al. (1998) Kidney Int. 54:590-602; Dragun et al. (1998) Kidney Int. 54:2113-22). Accordingly, the present invention contemplates the use of RNAi constructs in the above-described diseases.

In other embodiments, the subject RNAi constructs are selected by their ability to inhibit expression of a gene(s) essential for proliferation of smooth muscle cells or other cells of endothelium of blood vessels, such as proliferating cells involved in neointima formation. In such embodiments, the subject method can be used as part of a treatment or prophylaxis for restenosis.

Merely to illustrate, RNAi constructs applied to the blood vessel endothelial cells after angioplasty can reduce proliferation of these cells after the procedure. Merely to illustrate, a specific example is an siRNA complementary to c-myc (an oncogene). Down-regulation of c-myc inhibits cell growth. Therefore, siRNA can be prepared by synthesizing the following oligonucleotides:

5′-UCCCGCGACGAUGCCCCUCATT-3′
3′-TTAGGGCGCUGCUACGGGGAGU-5′

All bases are ribonucleic acids except the thymidines shown in bold, which are deoxyribose nucleic acids (for more stability). Double-stranded RNA can be prepared by mixing the oligonucleotides at equimolar concentrations in 10 mM Tris-Cl (pH 7.0) and 20 mM NaCl , heating to 95° C., and then slowly cooling to 37° C. The resulting siRNAs can then be purified by agarose gel electrophoresis and delivered to cells either free or complexed to a delivery system such as a cyclodextrin-based polymer. For in vitro experiments, the effect of the siRNA can be monitored by growth curve analysis, RT-PCR or western blot analysis for the c-myc protein.

It is demonstrated that antisense oligodeoxynucleotides directed against the c-myc gene inhibit restenosis when given by local delivery immediately after coronary stent implantation (see, for example, Kutryk et al. (2002) J Am Coll Cardiol. 39:281-287; Kipshidze et al. (2002) J Am Coll Cardiol. 39:1686-1691). Therefore, the present invention contemplates delivering an RNAi construct against the c-Myc gene (i.e., c-Myc RNAi construct) to the stent implantation site with an infiltrator delivery system (Interventional Technologies, San Diego, Calif.). Preferably, the c-Myc RNAi construct is directly coated on stents for inhibiting restenosis. Similarly, the c-Myc RNAi construct can be delivered locally for inhibiting myointimal hyperplasia after percutaneous transluminal coronary angioplasty (PTCA) and exemplary methods of such local delivery can be found, for example, Kipshidze et al. (2001) Catheter Cardiovasc Interv. 54:247-56. Preferably, the RNAi constructs are chemically modified with, for example, phosphorothioates or phosphoramidate.

Early growth response factor-1 (i.e., Egr-1) is a transcription factor that is activated during mechanical injury and regulates transcription of many genes involved with cell proliferation and migration. Therefore, down-regulation of this protein may also be an approach for prevention of restenosis. The siRNA directed against the Egr-1 gene can be prepared by synthesis of the following oligonucleotides:

5′-UCGUCCAGGAUGGCCGCGGTT-3′
3′-TTAGCAGGUCCUACCGGCGCC-5′

Again, all bases are ribonucleic acids except the thymidines shown in bold, which are deoxyribose nucleic acids. The siRNAs can be prepared from these oligonucleotides and introduced into cells as described herein.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Enhanced Serum Stability of Modified DNA:RNA Constructs

Materials:

Pre-formed duplexes (all from Dharmacon):

siFAS [MW 13317.2 g/mol]

5′ GUGCAAGUGCCAACCAGACTT 3′
3′ TTCACGUUCACGUUUGGUGUG 5′

siFAS2 [MW 13475.1 g/mol]

5′ PGUGCAAGUGCAAACCAGACTT 3′
3′ TTCACGUUCACGUUUGGUCUGP 5′
where P = phosphate group

siEGFPb [MW 13323.1 g/mol]

5′ GACGUAAACGGCCACAAGUUC 3′
3′ CGCUGCAUUUGCCGGUGUUCA 5′

FL-pGL2 [MW 13838.55 g/mol]

5′ XCGUACGCGGAAUACUUCGATT 3′
3′ TTGCAUGCGCCUUAUGAAGCU 5′
where X = fluorescein

Single strands

EGFPb-ss-sense (Dharmacon) [MW 6719.2 g/mol]

RNA, phosphodiester
5′ GACGUAAACGGCCACAAGUUC 3′

EGFPb-ss-antisense (Dharmacon)

RNA, phosphodiester
5′ ACUUGUGGCCGUUUACGUCGC 3′

JH-1 (Caltech Oligo Synthesis Facility)

DNA, phosphorothioate
5′ GACGTAAACGGCCACAAGTTCX 3′
where X = TAMRA

jhDNAs-1 (Caltech Oligo Synthesis Facility)

DNA, phosphodiester
5′ GACGTAAACGGCCACAAGTTC 3′

jhDNAs-2 (Caltech Oligo Synthesis Facility)

DNA, phosphodiester
5′ GACGTAAACGGCCACAAGTTCX 3′
where X = TAMRA

Duplex Formation (Annealing):

Duplexes were formed according to Dharmacon's recommended protocol. In short, one volume of the sense strand (50 μM) was combined with one volume of the antisense strand (50 μM) and one-half volume 5^{x} reaction buffer (100 mM KCl, 30 mM HEPES-KOH pH 7.5, 1.0 mM MgCl₂). The reaction mixture was heated to 90° C. for 1 min to denature strands, incubated at 37° C. for 1 h to allow annealing, and then stored at −20° C. Annealed duplexes were confirmed by gel electrophoresis (15% TBE gel).

In Vitro Mouse Serum Stability Results:

The stability of duplexes upon exposure to mouse serum (not heat-inactivated) was examined by gel electrophoresis. Ten microliters of 5 μM duplex was added to an equal volume of DNase-, RNase-free water or active mouse serum (Sigma) and incubated at 37° C. for 4 h. After this incubation, half of the volume (10 μL) was added to an equal volume of 5 mg/mL heparan sulfate (Sigma, in H₂O) and incubated at room temperature for 5 min. Four microliters of loading buffer was added to each 20-μL solution, and the resulting 24-μL solutions were loaded into wells of a 10-well, 15% TBE gel and electrophoresed at 100 V for 75 min. After electrophoresis, gels were incubated in 50 mL 0.5 μg/mL ethidium bromide (in 1×TBE buffer) for 30 min at room temperature and then photographed.

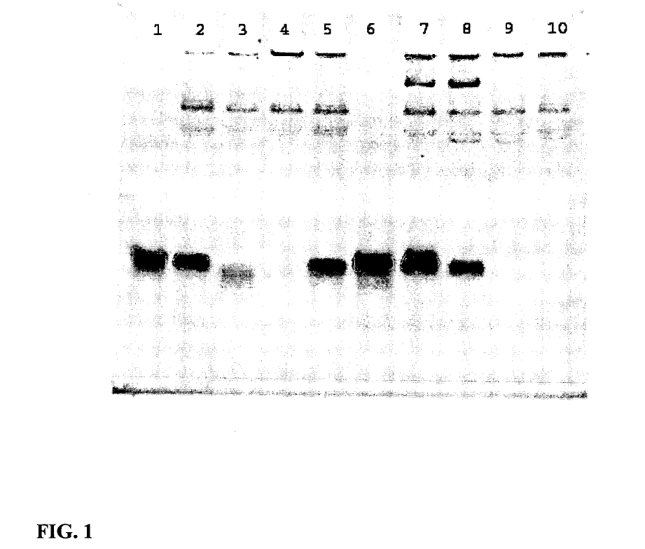

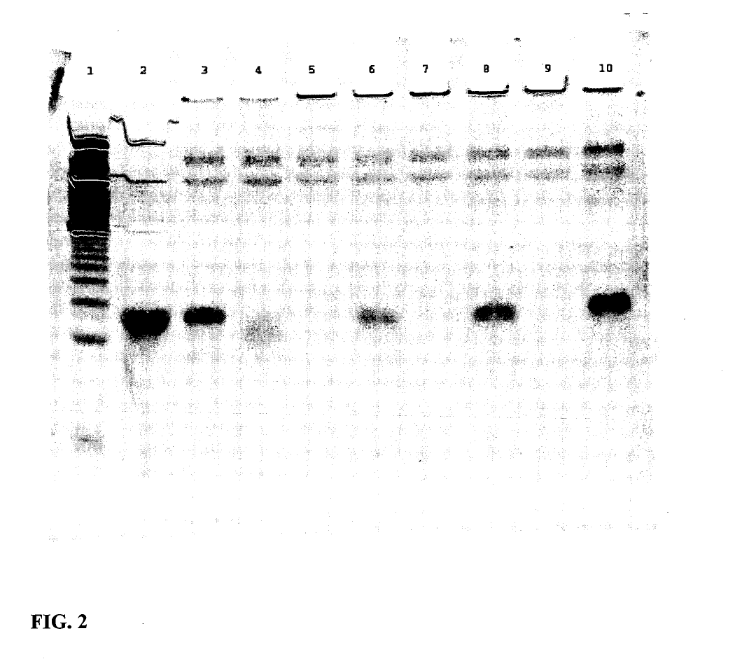

Our results indicated that siFAS2 showed near complete degradation by 4 hours of contact in 90% mouse serum while the hybrid JH-1:EFGPb-ss-antisense shows essentially no degradation. See FIG. 1 and FIG. 2

### Example 2

### Improved In Vivo Uptake of DNA:RNA Constructs

Each of four mice were injected with 2.5 mg/kg duplex via HPTV as indicated below:

| ID | Duplex |
|---|---|
| F1 | siFAS2 (unlabeled), naked |
| G1 | FL-pGL2 (5′ fluorescein), naked |
| M1 | JH-1: EGFPb-anti (3′ TAMRA), naked |

N1 JH-1:EGFPb-anti (3′TAMRA), CDP-Imid, 20:80 AdPEGLac:AdPEG 24 h post-injection, mice were sacrificed and livers were harvested, immersed in O.C.T. cryopreservation compound, and stored at −80° C. Morgan (Triche lab) kindly prepared thin sections (no fixative or counterstain added) which were examined immediately by confocal microscopy.

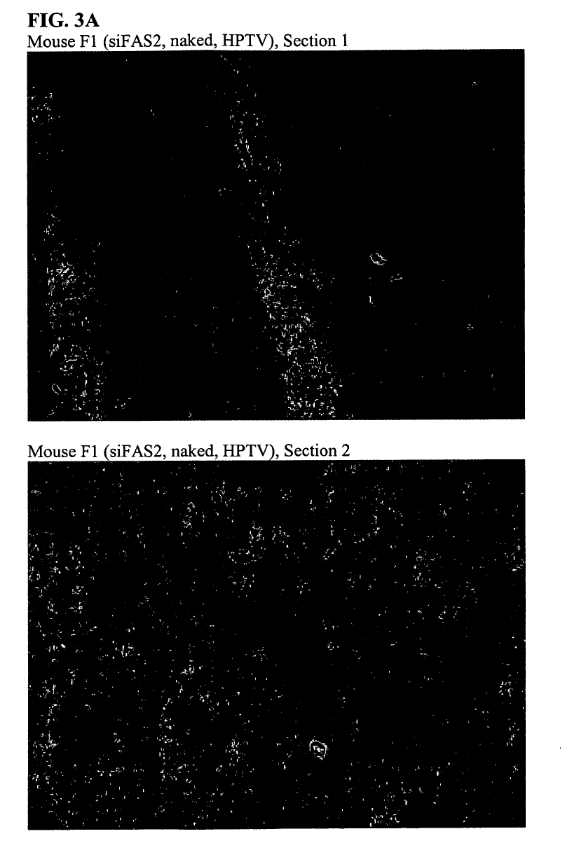

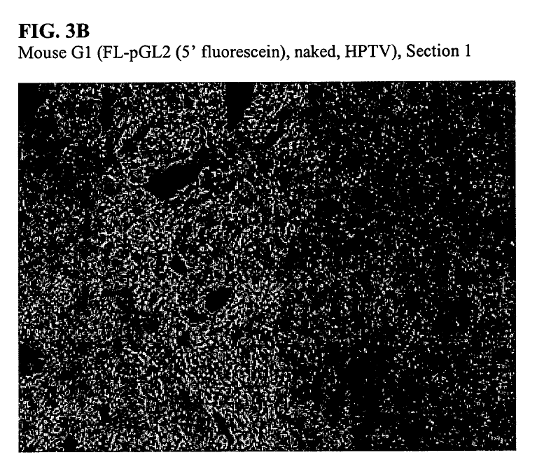

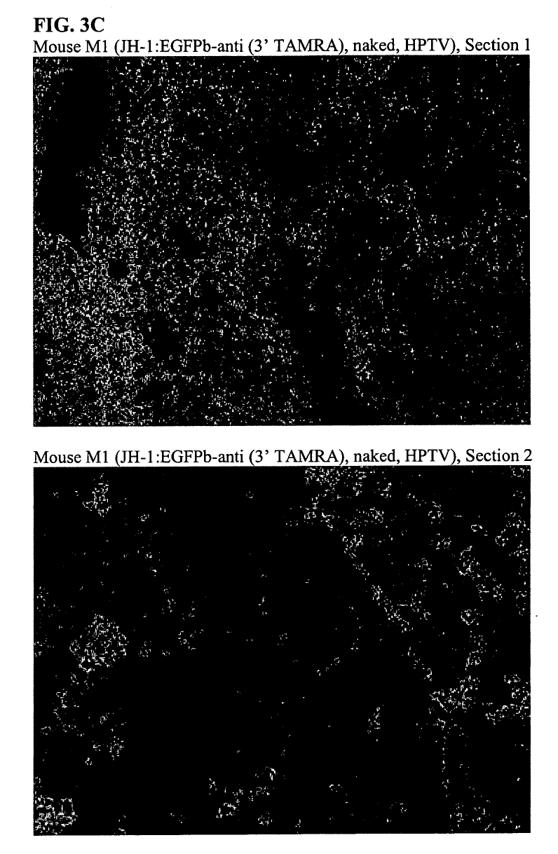

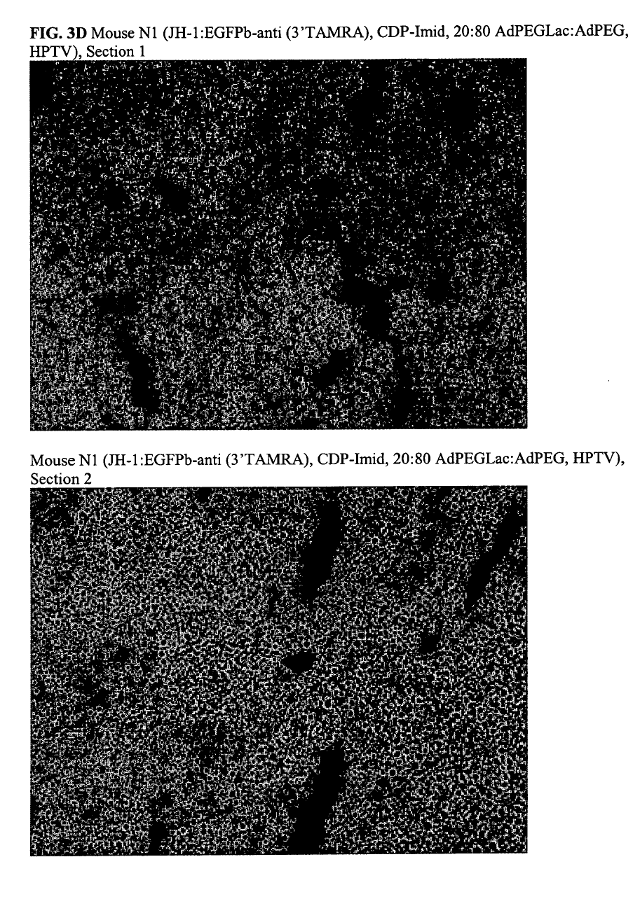

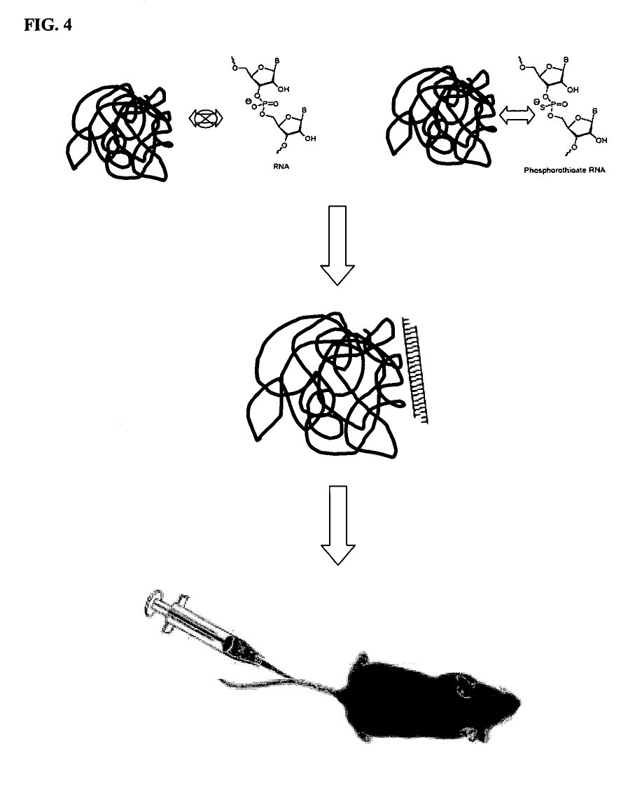

At 24 hours post injection, there is no fluorescence in the liver from injection of either F1 and G1 while significant fluorescence is observed in the liver from injections with M1. See FIG. 3A-3D.

### Example 3

### In vivo Delivery of a Phosphorothioate-Modified siRNA Duplex by Binding to an Asialofetuin Parrier protein

An siRNA duplex (RNA:RNA) against the luciferase gene was created by annealing a sense strand containing a phosphorothioate-modified backbone with an unmodified antisense strand (the strand with*denotes the phosphorothioate-modified sense strand).

*5′-CTTACGCTGAGTACTTCGAdTdT-3′*
3′-dTdTGAAUGCGACUCAUGAAGCU-5′

The sequence chosen is identical to the siGL3 duplex designed by Dharmacon to specifically target the luciferase gene.

Equimolar amounts of the modified siRNA duplex and asialofetuin (AF) protein were mixed in water and allowed to incubate at room temperature for 30 minutes. A control mixture was created containing only AF in water. After the incubation, 10% glucose in water was added in a 1:1 v/v ratio to each mixture, yielding a 5% glucose solution suitable for injection. The final dose of siRNA was 2.5 mg/kg body weight. The solutions were delivered by low-pressure tail-vein injection (0.15 mL per 20 g body weight) into transgenic C57BL/6 mice whose livers constitutively and stably express luciferase. See FIG. 4 for a schematic of this process.

Luciferase signal was monitored for three consecutive days using an in vivo IVIS 100 bioluminescence/optical imaging system. D-luciferin (Xenogen) dissolved in PBS was injected intraperitoneally at a dose of 150 mg/kg 10 min before measuring the light emission. General anesthesia was induced with 5% isoflurane and continued during the procedure with 2.5% isoflurane introduced via a nose cone. The signal intensity was quantified using IVIS Living Image software to integrate the photon flux from each mouse.

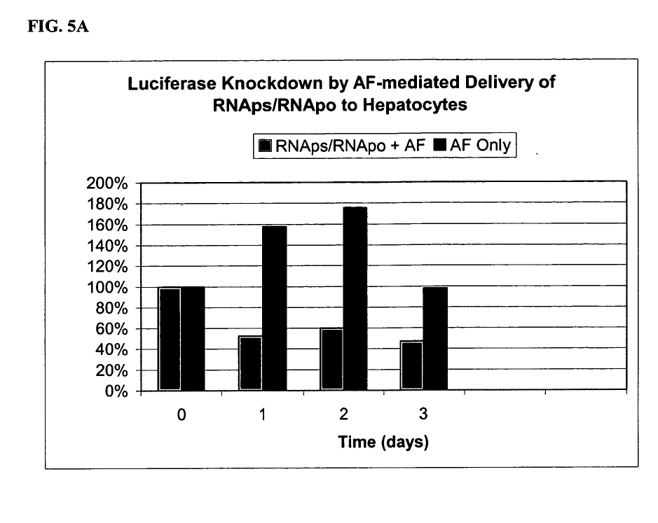

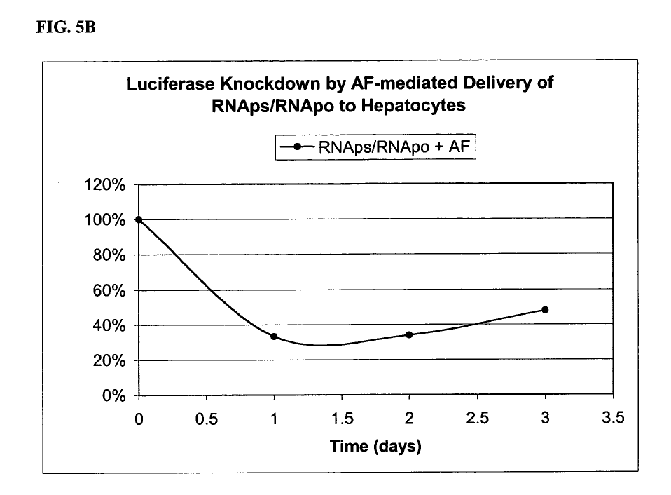

The data show that the siRNA construct was efficiently delivered to the targeted cells in vivo. See FIGS. 5A-B.

### Example 4

### Aptamer-siRNA Conjugate Stability and Structure Modelling

Recently, Farokhzad et al. (Farokhzad, O. C. et al. Nanoparticle-aptamer bioconjugates: a new approach for targeting prostate cancer cells. Cancer Research 64, 7668-7672 (2004)) have demonstrated the use of controlled release polymer nanoparticles targeted to prostate cancer cells through an RNA aptamer (xPSM-A10-3) developed by Lupold et al (Lupold, S. E., Hicke, B. J., Lin, Y. & Coffey, D. S. Identification and characterization of nuclease-stabilized RNA molecules that bind human prostate cancer cells via the prostate-specific membrane antigen. Cancer Research 62, 4029-4033 (2002)). This aptamer targets the prostate-specific membrane antigen (PSMA) that is overexpressed on prostate acinar epithelial cells. The aptamer system disclosed by Lupold et al. is utilized to demonstrate the instant methods.

Since one embodiment of the instant invention is the conjugation of an aptamer directly to a therapeutic molecule, such as an RNAi construct, without the need for a separate delivery vehicle, the investigation of the stability and structure of such an aptamer-siRNA conjugate was undertaken. These experiments indicate that it is possible for a hybrid aptamer-siRNA molecule to retain the activity of its aptamer and siRNA components. The xPSM-A10-3 aptamer to target the PSMA on LNCaP prostate cancer cells was chosed because its function has already been demonstrated in vitro and it was created specifically with 2′-F modified pyrimidines to provide enhanced stability. This is useful when moving into in vivo systems if this molecule is to be delivered systemically.

The following is the sequence of the xPSM-A10-3 aptamer:

5′-GGGAGGACGAUGCGGAUCAGCCAUGUUUACGUCACUCCUUGUCAAUC
CUCAUCGGC-3′

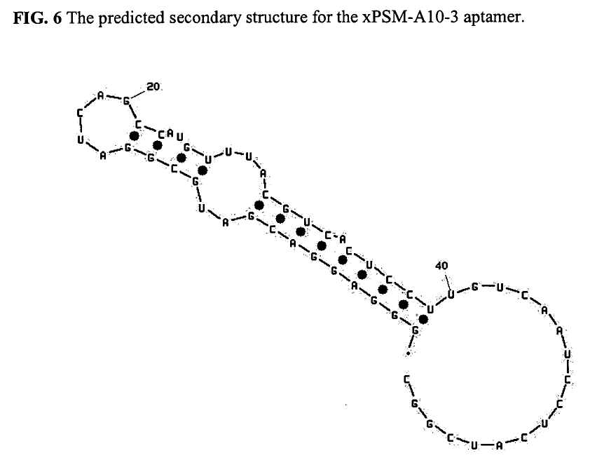

The Mfold web server for nucleic acid folding and hybridization prediction developed by M. Zuker (see Zuker, M. Mfold web server for nucleic acid folding and hybridization prediction. Nucleic Acids Research 31, 3406-3415 (2003)) gave the secondary structure for this aptamer as that shown in FIG. 6.

In this embodiment of the invention, the aptamer-siRNA conjugate also contains the sense strand from the siGL3 molecule developed by Dharmacon to target and degrade mRNA from the luciferase reporter gene. The following sequence was added to the 3′ end of the xPSM-A10-3 aptamer:

5′-AACUUACGCUGAGUACUUCGAUU-3′

The combination of the xPSM-A10-3 and siGL3 sequences yielded the following for the sense strand of this aptamer-siRNA conjugate (xPSM-A10-3-siGL3):

5′-GGGAGGACGAUGCGGAUCAGCCAUGUUUACGUCACUCCUUGUCAAUC
CUCAUCGGCAACUUACGCUGAGUACUUCGAUU-3′

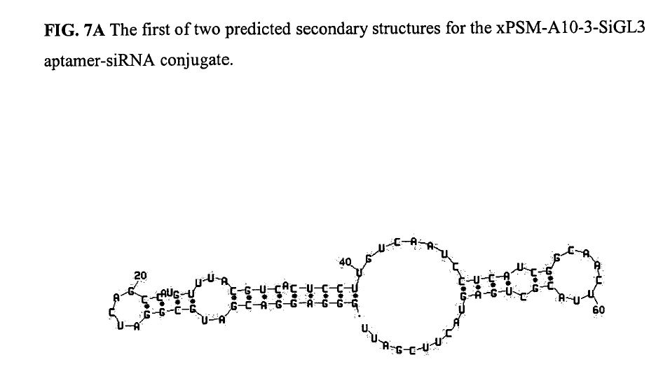

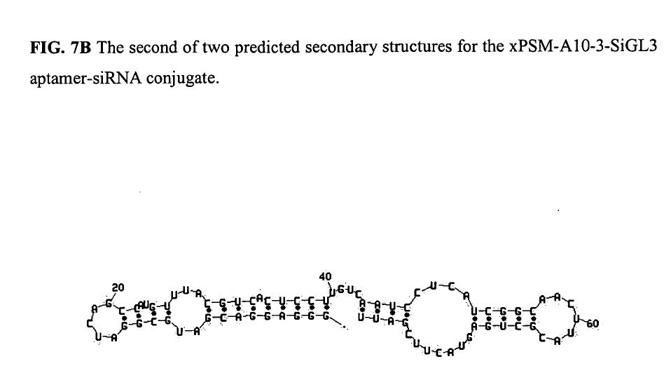

The aptamer sequence is at the 5′ end and the siGL3 sense strand is located at the 3′ end. The Mfold web server calculated the two most thermodynamically favorably secondary structures of this hybrid molecule, and these are depicted in FIGS. 7A-B.

The calculations show that the same basic secondary structure will again be adopted by the aptamer-siRNA conjugate as the original xPSM-A10-3 aptamer. The xPSM-A10-3 single-stranded molecule will need to be annealed to the antisense strand of the siGL3 duplex (5′-AAUCGAAGUACUCAGCGUAAGUU-3′). This will lead to a duplex region from nucleotides 60-77 on the xPSM-A10-3-siGL3 sequence given previously. The interaction of these two strands and the resulting secondary structure were modeled using PairFold (see Andronescu, M., Aguirre-Hemandez, R., Condon, A. & Hoos, H. H. RNAsoft: a suite of RNA secondary structure prediction and design software tools. Nucleic Acids Research 31, 3416-3422 (2003)). The following is the output given using dot-parenthesis notation in which a matching pair of parentheses represents a base pair and a dot represents an unpaired base:

5′-GGGAGGACGAUGCGGAUCAGCCAUGUUUACGUCACUCCUUGUCAAUCCUCAUCGGCAACUUACGCUGAGUACUUCGAUU AAUCGAAGUACUC
AGCGUAAGUU-3′
(((((((((..((((.....))..))...)))).))))).................((((((((((((((((((((((()))))))))))))))))))))))

Comparison of this predicted structure to those shown in FIGS. 5A-B for the xPSM-A10-3-siGL3 conjugate alone show that siGL3 duplex formation at the 3′ end has no effect on the secondary structure of the aptamer at the 5′ end.

The siGL3 duplex will likely still be able to function when attached to the 3′ end of the aptamer sequence. Several pieces of evidence support the notion that both the aptamer and the siGL3 duplex will remain functional. First, as seen in the above figures, the predicted secondary structure of the aptamer remains very similar whether or not it has the siGL3 sense sequence attached to its 3′ end. Second, aptamers have already been shown to retain their function even when attached to PEG chains on the surfaces of nanoparticles (see Farokhzad, O. C. et al. Nanoparticle-aptamer bioconjugates: a new approach for targeting prostate cancer cells. Cancer Research 64, 7668-7672 (2004)). Third, 5′ modifications on the sense strands of siRNA duplexes appear to have no effect on the gene silencing efficiency of the duplexes (see Manoharan, M. RNA interference and chemically modified small interfering RNAs. Current Opinion in Chemical Biology 8, 570-579 (2004)). The aptamer sequence can be viewed as a 5′ modification of the siGL3 duplex, and the siGL3 antisense strand remains unchanged.

These data demonstrate that it is possible to design an RNA molecule targeted by an aptamer sequence at the 5′ end and containing an siRNA duplex at the 3′ end. Such a molecule can be chemically modified to be stable in serum for in vivo delivery. Its small size (˜30 kDa) will allow good tissue penetration, rapid clearance from the blood, and urinary excretion (see Hicke, B. J. & Stephens, A. W. Escort aptamers: a delivery service for diagnosis and therapy. The Journal of Clinical Investigation 106, 923-928 (2000)). Moving to an in vivo system can be accomplished following initial in vitro studies performed by comparing uptake and luciferase downregulation between two cell lines that constitutively express luciferase: PSMA-positive LNCaP-LUC cells and PSMA-negative PC3-LUC cells. Luciferase downregulation will only be seen if the siGL3 duplex can reach the cytoplasm of the cells and still function despite the presence of the aptamer on the 5′ end of the sense strand. Comparison of the luciferase knockdown in LNCaP-LUC cells versus PC3-LUC cells will reveal the ability of the aptamer to increase uptake of the aptamer-siRNA conjugate through its binding to the PSMA. These experiments can be adapted for the creation of such molecules through an automated system that could be custom-made to deliver siRNA to potentially any protein or small molecule target.

1. A double-stranded nucleic acid for inhibiting expression of a target gene by an RNA interference mechanism, comprising:
a) a sense polynucleotide strand comprising one or more modifications or modified nucleotides;
b) an antisense polynucleotide strand, optionally comprising one or more modifications, having a designated sequence that hybridizes to at least a portion of a transcript of the target gene and is sufficient to inhibit expression of the target gene; and
c) an aptamer that binds to a preselected target.

2. The double-stranded nucleic acid of claim 1, wherein the sense polynucleotide comprises one or more modifications.

3. The double-stranded nucleic acid of claim 1, wherein the antisense polynucleotide comprises one or more modifications.

4. The double-stranded nucleic acid of claim 1, wherein the one or more modifications increase the isoelectric pH (pI) of the double-stranded nucleic acid relative to an unmodified double-stranded nucleic acid having the designated sequence by at least 0.5 units.

5. The double-stranded nucleic acid of claim 1, wherein the sense strand comprises at least 50% modified nucleotides.

6. The double-stranded nucleic acid of claim 1, wherein 50% or fewer of the nucleotides of the antisense polynucleotide are modified nucleotides.

7. The double-stranded nucleic acid of claim 2, wherein the one or more modifications increase the hydrophobicity of the double-stranded nucleic acid relative to an unmodified double-stranded nucleic acid having the designated sequence.

8. The double-stranded nucleic acid of claim 3, wherein the one or more modifications increase the hydrophobicity of the double-stranded nucleic acid relative to an unmodified double-stranded nucleic acid having the designated sequence.

9. The double-stranded nucleic acid of claim 1, wherein the double-stranded nucleic acid is a hairpin nucleic acid that is processed to an siRNA inside a cell, wherein the hairpin nucleic acid comprises a duplex portion, a loop portion and optionally a 3′ and/or 5′ tail portion.

10. The double-stranded nucleic acid of claim 1, wherein the double-stranded portion of the nucleic acid is 19-100 base pairs long.

11. The double-stranded nucleic acid of claim 1, wherein the double-stranded nucleic acid is internalized by cultured cells in the presence of 10% serum to a steady state level that is at least twice that of the unmodified double-stranded nucleic acid having the same designated sequence.

12. The double-stranded nucleic acid of claim 1, wherein the double-stranded nucleic acid has a serum half-life in a human or mouse of at least twice that of the unmodified double-stranded nucleic acid having the same designated sequence.

13. The double-stranded nucleic acid of claim 1, wherein the aptamer is associated with the sense strand.

14. The double-stranded nucleic acid of claim 13, wherein the aptamer is associated with the 5′ end of the sense strand.

15. The double-stranded nucleic acid of claim 9, wherein the aptamer is positioned within a portion selected from the group consisting of: the duplex portion, the loop portion, the 3′-tail or the 5′-tail.

16. The double-stranded nucleic acid of claim 1, wherein the preselected target is selected from the group consisting of: a serum protein, a membrane protein and a cell surface protein.

17. The double-stranded nucleic acid of claim 16, wherein the preselected target is internalized by cells.

18. The double-stranded nucleic acid of claim 16, wherein the serum protein is human serum albumin.

19. A pharmaceutical preparation for delivery of an RNAi nucleic acid to an organism, the composition comprising a pharmaceutically acceptable carrier and a double-stranded nucleic acid, comprising:
a) a sense polynucleotide strand comprising one or more modifications to the sugar-phosphate backbone; and
b) an RNA antisense polynucleotide strand having a designated sequence that hybridizes to at least a portion of a transcript of a target gene and is sufficient to inhibit expression of the target gene,
wherein the one or more modifications to the sugar-phosphate backbone increase non-covalent association of the double-stranded nucleic acid with one or more species of protein as compared to an unmodified double-stranded nucleic acid having the designated sequence.

20. The pharmaceutical preparation of claim 19, wherein the sense polynucleotide comprises one or more phosphorothioate modifications to the sugar-phosphate backbone.

21. The pharmaceutical preparation of claim 20, wherein the sense polynucleotide comprises greater than 50% phosphorothioate modifications.

22. The pharmaceutical preparation of claim 21, wherein the sense polynucleotide comprises 100% phosphorothioate modifications.

23. The pharmaceutical preparation of claim 19, wherein the sense polynucleotide is selected from the group consisting of: a sense polynucleotide strand and an antisense polynucleotide strand.

24. The pharmaceutical preparation of claim 19, wherein the preparation further comprises a polypeptide.

25. The pharmaceutical preparation of claim 24, wherein the polypeptide is selected from the group consisting of: a serum polypeptide and a cell targeting polypeptide.

26. The pharmaceutical preparation of claim 25, wherein the cell targeting polypeptide is a polypeptide comprising a plurality of galactose moieties.

27. The pharmaceutical preparation of claim 19, wherein the double stranded nucleic acid further comprises an aptamer.
